(19) United States
(12) Patent Application Publication
(10) Pub. No.: US 2026/0007600 A1
(43) Pub. Date: Jan. 8, 2026

(54) NUCLEIC ACID DRUG FOR TREATING HYPERURICEMIA-RELATED DISEASES, PREPARATION METHOD THEREFOR, AND USE THEREOF

(71) Applicant: Rigerna Therapeutics (Beijing) Co., Ltd., Beijing (CN)

(72) Inventors: Yuanyu HUANG, Beijing (CN); Mengjie ZHANG, Beijing (CN)

(73) Assignee: Rigerna Therapeutics (Beijing) Co., Ltd., Beijing (CN)

(21) Appl. No.: 18/993,016

(22) PCT Filed: Dec. 27, 2022
(86) PCT No.: PCT/CN2022/142382

§ 371 Date: Jan. 10, 2025

(51) Int. Cl. A61K 9/1271; A61K 9/00; A61K 9/1277; A61K 9/51; A61K 31/7105; A61K 38/00; A61P 3/00; C12N 9/06

(52) U.S. Cl. CPC A61K 9/1271; A61K 9/0019; A61K 9/1277; A61K 9/5123; A61K 9/5192; A61K 31/7105; A61P 3/00; C12N 9/0048; A61K 38/00; C12Y 107/03003

(57) ABSTRACT

Provided is an mRNA-liposome complex. The mRNA-liposome complex includes a liposome and a nucleic acid. The nucleic acid includes at least one mRNA encoding urate oxidase or recombinant urate oxidase. The liposome includes at least two lipids, and the at least two lipids include a core lipid. The liposome has a pKa ranging from 6.0 to 6.3. A molar percentage of the core lipid in a total molar amount of the liposome is not less than 15%.

### FIELD

The present disclosure belongs to the technical field of biopharmaceuticals. Specifically, the present disclosure relates to a nucleic acid drug for treating hyperuricemia-related diseases, a preparation method thereof, and use thereof. More specifically, the present disclosure relates to an mRNA-liposome complex and a preparation method thereof, a pharmaceutical composition, uses thereof, a method for treating and/or preventing hyperuricemia-related diseases, and a method for delivering mRNA mainly to liver cells.

### BACKGROUND

Hyperuricemia (HUA) is a chronic metabolic disease caused by purine metabolism disorder, which is a special metabolic state of a body caused by an excessively high blood uric acid level due to insufficient renal excretion, excessive secretion, or insufficient intestinal excretion. In the human body, uric acid is the final metabolite of purine. Due to the characteristic that uric acid is slightly soluble in water, it is easy to deposit in different tissues such as joints, cartilage, and kidneys in the form of urate crystals, thereby causing various diseases such as gout and nephritis.

Studies have found that among mammals, only humans, birds, and some primates use uric acid as the final metabolite of purine, while most other mammals can further decompose uric acid into water-soluble allantoin through urate oxidase (Uox) (also known as uricase) and excrete the allantoin from the body. During the long evolutionary process of the humans, Uox gene has undergone nonsense mutations, causing the Uox gene to become a pseudogene in the humans and no longer express functional proteins.

Currently, there are three main types of clinical anti-hyperuricemia drugs. The first type thereof inhibits a production of uric acid by inhibiting the activity of xanthine oxidase, thereby reducing a concentration of blood uric acid, such as allopurinol and febuxostat. The second type thereof reduces the concentration of blood uric acid by promoting uric acid excretion, such as benzbromarone. However, these small molecule drugs require lifelong medication and have key problems such as drug resistance, and may even cause fatal toxic side effects. The third type is urate oxidase drugs, which belong to protein therapy. The urate oxidase drugs are injected into body to mainly catalyze the slightly water-soluble uric acid into water-soluble allantoin, which is then excreted from the body. Currently, the urate oxidase drugs on the market still have problems such as short half-life and strong immunogenicity.

Compared with the protein therapy, there are several potential advantages of replacing therapeutic proteins with mRNA-mediated protein expression. mRNA may be expressed directly in the cytoplasm without entering the nucleus, thereby avoiding the risk of genomic integration. On the other hand, by appropriately modifying a regulatory sequence and optimizing a system, the stability and translation efficiency of mRNA can be significantly improved. The efficient delivery system enables mRNA to be rapidly taken up and expressed in the cytoplasm. At the same time, mRNA, as the smallest gene carrier, has low immunogenicity and will not be degraded due to antibody reaction. Thus, mRNA can be translated multiple times to improve efficiency. Compared with the proteins, the production, preparation, and purification process of mRNA is simpler, faster, and has lower production costs.

The effectiveness and safety of lipid nanoparticles have been well demonstrated in decades of nanomedicine research. The first marketed siRNA drug and the three recent mRNA-based COVID-19 vaccines (mRNA-1273, BNT162b2, and CVnCOV) all use the lipid nanoparticles as delivery vehicles.

Therefore, it is urgent to develop a safe and effective mRNA drug against hyperuricemia with high delivery efficiency.

### SUMMARY

In a first aspect of the present disclosure, the present disclosure provides an mRNA-liposome complex. The mRNA-liposome complex includes a liposome and a nucleic acid. The nucleic acid includes at least one mRNA, and the mRNA encodes urate oxidase or recombinant urate oxidase. The liposome includes at least two lipids including a core lipid. A molar percentage of the core lipid in a total molar amount of the liposome is not less than 15%.

In a second aspect of the present disclosure, the present disclosure provides a method for preparing the mRNA-liposome complex according to the first aspect. The method includes: mixing the liposome with the nucleic acid, to obtain the mRNA-liposome complex.

In a third aspect of the present disclosure, the present disclosure provides a pharmaceutical composition. The pharmaceutical composition includes the mRNA-liposome complex according to the first aspect and a pharmaceutically acceptable excipient or carrier.

In a fourth aspect of the present disclosure, the present disclosure provides a method for treating and/or preventing hyperuricemia-related diseases. The method includes administering a pharmaceutically acceptable amount of the mRNA-liposome complex according to the first aspect to a subject.

In a fifth aspect of the present disclosure, the present disclosure provides a method for delivering mRNA mainly to liver cells. The method includes: administering a pharmaceutically acceptable amount of the mRNA-liposome complex according to the first aspect to a subject.

### BRIEF DESCRIPTION OF THE DRAWINGS

FIG. 1 is a structural formula of core lipid A1-D1-5 in Example 2 according to the present disclosure.

FIG. 2 is an electron micrograph of lipid nanoparticles mUox@iLAND in Example 3 according to the present disclosure.

FIG. 3 shows stability of lipid nanoparticles mUox@iLAND in Example 3 according to the present disclosure.

FIG. 4 shows pKa of lipid nanoparticles mUox@iLAND in Example 4 according to the present disclosure.

FIG. 5 shows a flow cytometer analysis of cell entry of lipid nanoparticles in Example 5 according to the present disclosure.

FIG. 6 shows a laser confocal microscopy observation of cell entry of lipid nanoparticles in Example 5 according to the present disclosure.

FIG. 7 shows expression of Uox proteins in cells of mUox@iLAND group analyzed by Western blotting in Example 6 according to the present disclosure.

FIG. 8 shows expression of Uox proteins in cells of Uox1@iLAND group, Uox2@iLAND group, and Uox3@iLAND group analyzed by Western blotting in Example 6 according to the present disclosure.

FIG. 9 shows distribution of mUox@iLAND in animals observed by fluorescence imaging in Example 7 according to the present disclosure.

FIG. 10 shows expression of mUox@iLAND in animals observed by bioluminescent imaging in Example 7 according to the present disclosure.

FIG. 11 is quantitative results of fluorescence quantification and bioluminescence imaging in Example 7 according to the present disclosure.

FIG. 12 shows enrichment of mRNA in mUox@iLAND in the liver in Example 7 according to the present disclosure (6 hours).

FIG. 13 shows enrichment of mRNA in mUox@iLAND in the liver in Example 7 according to the present disclosure (24 hours).

FIG. 14 shows changes in a concentration of uric acid in serum of animals in respective groups before and after modeling in Example 8 according to the present disclosure.

FIG. 15 shows changes in a concentration of uric acid in serum of animals in respective groups during a treatment process in Example 8 according to the present disclosure.

FIG. 16 shows changes in weight of animals in respective groups during a treatment process in Example 8 according to the present disclosure.

FIG. 17 shows an analysis of serum biochemical indices of animals in respective groups at the end of treatment in Example 8 according to the present disclosure.

FIG. 18 shows changes in a concentration of uric acid in serum of animals in respective groups before and after modeling of step 1 in Example 9 according to the present disclosure.

FIG. 19 shows changes in a concentration of uric acid in serum of animals in respective groups during a treatment process of step 1 in Example 9 according to the present disclosure.

FIG. 20 shows changes in weight of animals in respective groups during a treatment process of step 1 in Example 9 according to the present disclosure.

FIG. 21 shows an analysis of serum biochemical indices of animals in respective groups at the end of treatment of step 1 in Example 9 according to the present disclosure.

FIG. 22 shows a metabolomics analysis of small molecule metabolism of animals in respective groups after treatment of step 1 in Example 9 according to the present disclosure.

FIG. 23 shows observation results of pathological sections of tissues of animals in respective groups of step 1 in Example 9 according to the present disclosure.

FIG. 24 shows changes in a concentration of uric acid in serum of animals in respective groups during a treatment process of step 2 in Example 9 according to the present disclosure.

### DETAILED DESCRIPTION

Embodiments of the present disclosure are described in detail below. The embodiments described below are exemplary and are only used to explain the present disclosure, and are not to be construed as limiting the present disclosure. Techniques or conditions that are not specified in the embodiments shall be performed in accordance with the techniques or conditions described in the literatures in the art or in accordance with the product instruction. Reagents or instruments without indicating the manufacturer are all common products that are commercially available.

It should be noted that, with respect to the structural formulas and chemical formulas described in the examples or embodiments of the present disclosure, the present disclosure is intended to cover all alternatives, modifications, and equivalent technical solutions, which all fall within the scope of the present disclosure as defined in the claims. One skilled in the art will recognize that many methods and materials similar or equivalent to those described herein may be used to practice the present disclosure. The present disclosure is not limited to the methods and materials described herein. In the event that one or more of the incorporated literature, patents, and similar materials differs from or contradicts the present disclosure, including, but not limited to, defined terms, term usage, described techniques, or the like, the present disclosure prevails.

It should be further recognized that certain features of the present disclosure, for the sake of clarity and visibility, are described in a plurality of separate examples or embodiments, but may also be provided in combination in a single example or embodiment. Conversely, various features of the present disclosure, for brevity, are described in a single example or embodiment, but may also be provided individually or in any suitable sub-combination.

Unless otherwise indicated, technical and scientific terms used in the present disclosure have the same meanings as commonly understood by those skilled in the art to which the present disclosure belongs. Unless otherwise noted, all patent publications cited in the entire content of the present disclosure are incorporated herein by reference in their entireties.

As used herein, the terms “include”, “contain”, or “comprise” are open expressions, that is, including the contents specified in the present disclosure but not excluding other contents.

As used herein, compounds of the present disclosure also include isotopically labeled compounds of the present disclosure, which are identical to those described herein except for the fact that one or more atoms are replaced by an atom having an atomic mass or mass number different from the naturally occurring atomic mass or mass number. Exemplary isotopes that may also be incorporated into the compounds of the present disclosure include isotopes of hydrogen, carbon, nitrogen, oxygen, phosphorus, sulfur, fluorine, and chlorine.

Compounds of the present disclosure and pharmaceutically acceptable salts of the compounds containing the aforementioned isotopes and/or other isotopes of other atoms are within the scope of the present disclosure. The compounds of the present disclosure may contain asymmetric or chiral centers. Therefore, the compounds of the present disclosure may be present in different stereoisomeric forms. It can be expected that all stereoisomeric forms of the compounds of the present disclosure, including but not limited to, diastereomers, enantiomers, atropisomers, and mixtures thereof such as racemic mixtures, shall fall within the scope of the present disclosure.

Unless otherwise indicated, the structures described herein are also meant to include all isomers (e.g., enantiomers, diastereomers, atropisomers, and geometric (or conformational) forms) of the structures. Therefore, single stereochemical isomers as well as enantiomeric mixtures, diastereomeric mixtures, and geometric (or conformational) mixtures of the compounds of the present disclosure are within the scope of the present disclosure.

Therefore, as described herein, the compounds of the present disclosure may exist in the form of one or a mixture of possible isomers, rotamers, atropisomers, tautomer, for example, in the form of substantially pure geometric (cis or trans) isomers, diastereomers, optical isomers (enantiomers), racemates, or mixtures thereof.

As used herein, the term “solvate” refers to an association between one or more solvent molecules and a compound of the present disclosure. Solvents for forming the solvates include, but are not limited to, water, isopropanol, ethanol, methanol, dimethyl sulfoxide, ethyl acetate, acetic acid, and aminoethanol. The term “hydrate” refers to an associated complex in which the solvent molecule is water.

As used herein, the term “pharmaceutically acceptable” means that the substance or composition must be chemically and/or toxicologically compatible with the other ingredients including the formulation and/or the mammal to be treated therewith.

As used herein, the term “pharmaceutically acceptable salts” refers to both organic and inorganic salts of the compounds of the present disclosure. The term “pharmaceutically acceptable salts” is well known in the art.

As used herein, the terms “optionally” or “optional” generally mean that the event or condition described subsequently may, but may not, occur, and the description includes situations in which the event or condition occurs, as well as situations in which it does not occur.

As used herein, the term “pharmaceutically acceptable excipient or carrier” includes any solvent, dispersion medium, coating material, surfactant, antioxidant, preservative (e.g., antibacterial agent, antifungal agent), isotonic agent, salt, drug stabilizer, binder, excipient, dispersant, lubricant, sweetener, flavoring agent, coloring agent, or a combination thereof, which are known to those skilled in the art (for example, as described in Remington's Pharmaceutical Sciences, 18th Ed. Mack Printing Company, 1990, pp. 1289-1329). Except where any conventional carrier is incompatible with the active ingredient, its use in the therapeutic or pharmaceutical compositions is contemplated.

As used herein, the term “treatment” refers to the use of drugs to obtain a desired pharmacological and/or physiological effect. The effect may be prophylactic, in terms of complete or partial prevention of the disease or its symptoms, and/or therapeutic, in terms of partial or complete cure of the disease and/or adverse effects resulting from the disease. As used herein, the term “treatment” covers the treatment of diseases in mammals, in particular diseases in humans, including: (a) preventing the occurrence of a disease or condition in an individual who is susceptible to the disease but has not yet been diagnosed with the disease; (b) inhibiting the disease, such as arresting the progression of the disease; or (c) alleviating the disease, such as alleviating the symptoms associated with the disease. As used herein, the term “treatment” encompasses any administration of a drug or compound to a subject to treat, cure, alleviate, ameliorate, mitigate, or inhibit a disease in the subject, including but not limited to, administering a drug containing a compound described herein to a subject in need thereof.

As used herein, terms “hyperuricemia-related diseases” and “related diseases caused by elevated uric acid level” are synonymous, both referring to diseases caused by increased uric acid levels, including but not limited to, hyperuricemia, gout, or other related diseases caused by elevated uric acid level (such as gouty nephropathy, gouty vasculopathy, gouty cardiomyopathy, etc.).

The present disclosure aims to solve one of the technical problems in the related art at least to a certain extent. To this end, one object of the present disclosure is to provide a nucleic acid drug that can effectively treat hyperuricemia or other related diseases caused by elevated uric acid.

mRNA-Liposome Complex

In a first aspect of the present disclosure, the present disclosure provides an mRNA-liposome complex. The mRNA-liposome complex includes a liposome and a nucleic acid. The nucleic acid includes at least one mRNA. The mRNA encodes urate oxidase (UOX) or recombinant urate oxidase. The liposome includes at least two lipids, which include a core lipid. A molar percentage of the core lipid in a total molar amount of the liposome is not less than 15%.

According to an embodiment of the present disclosure, the core lipid is a main skeleton of the mRNA-liposome complex and ensures that the mRNA-liposome complex can well pass through the cell membrane. The mRNA-liposome complex is present as neutral in the peripheral environment of the human body (blood, tissue fluid), and it has low toxicity and strong biocompatibility. In addition, the mRNA-liposome complex can be ionized in the acidic environment (endosome/lysosome) within the cell, and thus can exert an excellent endosomal escape effect, thereby effectively releasing mRNA and improving the therapeutic effect of mRNA. Therefore, the above-mentioned mRNA-liposome complex can efficiently deliver mRNA into the animal body and effectively pass through the cell membrane with the improved cell entry efficiency of mRNA, thereby increasing the expression level of the protein. Compared with a mRNA-liposome complex without containing the above-mentioned core lipid or with the molar amount of the above-mentioned core lipid less than 15%, the cell entry efficiency of the mRNA-liposome complex of the present disclosure is improved by at least 20%. Furthermore, the mRNA-liposome complex of the present disclosure has excellent biocompatibility and degradability and has no obvious toxic side effects, thereby having good clinical application prospects.

In some optional embodiments of the present disclosure, the mRNA-liposome complex may further include at least one of the following technical features.

In some optional embodiments of the present disclosure, the liposome includes the core lipid, an auxiliary lipid, a steroid, and a PEG lipid; and a molar ratio of the core lipid, the auxiliary lipid, the steroid, and the PEG lipid is (20 to 60):(10 to 50):(30 to 50):(0.5 to 2.5).

In some optional embodiments of the present disclosure, the liposome has a pKa ranging from 6.0 to 6.3. In this way, it is ensured that the mRNA-liposome complex is present as neutral in the human peripheral environment (blood, tissue fluid), and has low toxicity and strong biocompatibility. In addition, the mRNA-liposome complex can be ionized in the acidic environment (endosome/lysosome) within the cell, and thus may exert an excellent endosomal escape effect, thereby effectively releasing mRNA and improving the therapeutic effect of mRNA.

In some optional embodiments of the present disclosure, the core lipid is selected from a compound having a structure represented by Formula (I) or a stereoisomer, tautomer, solvate, and pharmaceutically acceptable salt thereof,

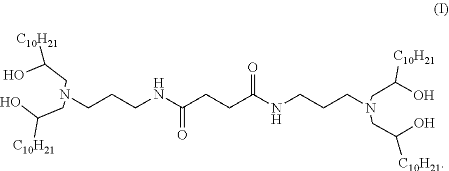

In some optional embodiments of the present disclosure, the mRNA is used to encode a protein that can be expressed in the liver. The inventors have found through experiments that, compared with traditional liposome drugs, the mRNA-liposome complex of the present disclosure can efficiently deliver nucleic acid molecules such as mRNA to the liver or enable the nucleic acid molecules such as mRNA to mainly target to the liver, and can also increase the content or expression of the nucleic acid molecules such as mRNA in the liver.

As used herein, the term “target” is non-specific. The term “target to the liver” means that mRNA may be delivered to the liver, and the mRNA may express the corresponding protein in large quantities in the liver. The protein mainly exerts biological activity in the liver. The targeting of the liver is non-specific. The mRNA may also be delivered to other organs of the body except the liver to express the corresponding protein, but it is mainly delivered to the liver.

In some optional embodiments of the present disclosure, the urate oxidase (UOX) or recombinant urate oxidase has an activity in reducing the uric acid level. Therefore, the mRNA-liposome complex of the present disclosure may deliver mRNA to the liver, achieve large-scale expression of the recombinant urate oxidase or urate oxidase in the liver, and maintain the uric acid level of patients with hyperuricemia at a normal level. Moreover. the efficacy of the mRNA-liposome complex of the present disclosure may last for several weeks, which is superior to commercially available recombinant urate oxidase drugs (such as Rasburicase).

As used herein, the term “recombinant urate oxidase” refers to a recombinant protein containing a functional fragment of urate oxidase. The “functional fragment” refers to a partial or complete fragment of the urate oxidase, which can maintain the biological activity of the urate oxidase, that is, the biological activity of decomposing the uric acid into allantoin.

Those skilled in the art can understand that the recombinant protein refers to a protein obtained by using recombinant DNA or recombinant RNA technology.

For example, the urate oxidase is selected from at least one of mouse, primate, bovine, horse, dairy cow, pig, sheep, goat, dog, cat, rabbit, and camel origins, and preferably, from at least one of mouse and primate origins.

In some optional embodiments of the present disclosure, the urate oxidase or recombinant urate oxidase has an amino acid sequence as set forth in any one of SEQ ID NO: 1 to SEQ ID NO: 6, or an amino acid sequence having at least 90% homology to the amino acid sequence as set forth in any one of SEQ ID No: 1 to SEQ ID No: 6. Preferably, the urate oxidase has an amino acid sequence having at least 91%, 92%, 93%, 94%, 95%, 96%, 97%, 98%, or 99% homology to the amino acid sequence as set forth in any one of SEQ ID No: 1 to SEQ ID No: 6.

In some optional embodiments of the present disclosure, the mRNA has a nucleotide sequence as set forth in any one of SEQ ID No: 7 to SEQ ID No: 12, or a nucleotide sequence having at least 90% sequence similarity (similarity or identity) to the nucleotide sequence as set forth in any one of SEQ ID No: 7 to SEQ ID No: 12. Preferably, the mRNA has a nucleotide sequence having at least 91%, 92%, 93%, 94%, 95%, 96%, 97%, 98%, or 99% similarity to the nucleotide sequence as set forth in any one of SEQ ID No: 7 to SEQ ID No: 12. Optionally, the similarity of the mRNA to the nucleotide sequence as set forth in any one of SEQ ID No: 7 to SEQ ID No: 12 is that the nucleotide sequence the mRNA differs from the nucleotide sequence as set forth in any one of SEQ ID No: 7 to SEQ ID No: 12 by 1 to 15 (preferably, 1 to 10) nucleotides.

As used herein, the nucleotide sequence as set forth in any one of SEQ ID No: 7 to SEQ ID No: 12 is a coding region (CDS) sequence in the mRNA. It should be noted that the expression of “the mRNA has a nucleotide sequence as set forth in any one of SEQ ID No: 7 to SEQ ID No: 12” means that the mRNA may further contain a sequence of a non-coding region in addition to the nucleotide sequence as set forth in any one of SEQ ID No: 7 to SEQ ID No: 12. The sequence of the non-coding region is not specifically limited, and all the mRNAs containing the nucleotide sequence as set forth in any one of SEQ ID No: 7 to SEQ ID No: 12 shall fall within the protection scope of the present disclosure.

As used herein, the terms “identity”, “homology”, “similarity”, or “concordance” are all used to describe a percentage of identical amino acids or nucleotides between two amino acid or nucleic acid sequences determined by conventional methods relative to an reference amino acid or nucleic acid sequence, for example, see Ausubel et al., eds. (1995), Current Protocols in Molecular Biology, Chapter 19 (Greene Publishing and Wiley-Interscience, New York); and the ALIGN program (Dayhoff (1978), Atlas of Protein Sequence and Structure 5: Suppl. 3 (National Biomedical Research Foundation, Washington, DC).

As used herein, the term “at least 90% sequence homology” or “at least 90% sequence similarity” refers to at least 90%, for example, 90%, 91%, 92%, 93%, 94%, 95%, 96%, 97%, 98%, 99%, 99.5%, or 99.9% homology or similarity to a respective reference sequence.

In some optional embodiments of the present disclosure, 5′ end of the mRNA is linked to 5′UTR; and/or 3′ end of the mRNA is linked to 3′UTR.

For example, the 5′UTR has a nucleotide sequence as set forth in SEQ ID NO: 13.

For example, the 3′UTR has a nucleotide sequence as set forth in SEQ ID NO: 14.

In some optional embodiments of the present disclosure, the molar ratio of the core lipid to the total lipid is not less than 15% and not more than 45%. Preferably, a molar ratio of the core lipid, the auxiliary lipid, the steroid, and the PEG lipid is (20 to 60):(10 to 50):(30 to 50):(0.5 to 2.5). Therefore, the delivery capability of mRNA molecules to the liver can be further improved, and the cell entry efficiency of mRNA and the level expression of the protein can be further improved. In particular, when the mRNA encodes the recombinant urate oxidase or urate oxidase, the mRNA can be mainly delivered to the liver, which achieves high expression of the recombinant urate oxidase or urate oxidase in the liver. Further, the uric acid may be decomposed into allantoin, and hyperuricemia can be effectively treated. Compared with those mRNA-liposome complexes without the core lipid, the presence of the core lipid (when the molar ratio of the core lipid in the total lipid is not less than 15%) allows the cell entry efficiency of the mRNA-liposome complex to increase by at least 20%.

As used herein, the term “mainly delivered” means that after the mRNA-liposome complex is administered via a systemic route, the majority of the mRNA is delivered to liver cells, where the majority means at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%.

In some optional embodiments of the present disclosure, the molar ratio of the core lipid, the auxiliary lipid, the steroid, and the PEG lipid is (30 to 39):(9 to 15):(40 to 50):(0.5 to 1).

For example, the molar ratio of the core lipid, the auxiliary lipid, the steroid, and the PEG lipid is (30 to 39):(9 to 11):(40 to 45):(0.5 to 0.8).

In some preferred embodiments of the present disclosure, the molar ratio of the core lipid, the auxiliary lipid, the steroid, and the PEG lipid is a molar percentage, that is, the molar ratios of the core lipid, the auxiliary lipid, the steroid, and the PEG lipid adds up to 100%.

In some optional embodiments of the present disclosure, the auxiliary lipid may be selected from those suitable species well known in the art. For example, the auxiliary lipid is selected from at least one of 1,2-distearoyl-sn-glycero-3-phosphocholine (DSPC), 1,2-dipalmitoyl-sn-glycero-3-phosphocholine (DPPC), 1,2-dipalmitoyl-sn-glycero-3-phosphoethanolamine (DPPE), 1,2-dimyristoyl-sn-glycero-3-phosphocholine (DMPC), 1,2-dimyristoyl-sn-glycero-3-phosphoethanolamine (DMPE), 1-palmitoyl-2-oleoyl-sn-glycero-3-phosphocholine (POPC), 1,2-dioleoyl-sn-glycero-3-phosphocholine (DOPC), 2-dioleoyl-sn-glycerol-3-phospho-(1′-rac-glycerol) (DOPG), 1,2-dioleoyl-sn-glycero-3-phosphoethanolamine (DOPE), sphingomyelin (SM), and diethyl pyrocarbonate (DEPC).

In some optional embodiments of the present disclosure, the steroid is selected from at least one of cholesterol, coprosterol, sitosterol, ergosterol, campesterol, stigmasterol, and brassicasterol, and preferably, cholesterol.

In some optional embodiments of the present disclosure, the PEG lipid is selected from at least one of 2-[(polyethylene glycol)-2000]-N,N-tetracosyl acetamide (ALC-0159), 1,2-dimyristoyl-sn-glycero methoxy polyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmitoyl, PEG-dioleyl, PEG-distearyl, PEG-diacylglycerolamide (PEG-DAG), PEG-dipalmitoyl phosphatidylethanolamine (PEG-DPPE), PEG-phosphatidylethanolamine (PEG-PE), PEG-succinic diacylglycerol (PEG-S-DAG), PEG-ceramide (PEG-cer), PEG-dialkoxypropyl carbamate, and PEG-1,2-dimyristoyloxypropyl-3-amine (PEG-c-DMA).

In some optional embodiments of the present disclosure, the PEG lipid is selected from at least one of DMG-PEG 2000, DSPE-PEG 2000, DPPE-PEG 2000, and DMA-PEG 2000.

In some optional embodiments of the present disclosure, the mass ratio of the liposome to the mRNA is (1 to 30):1, preferably (10 to 30):1, and more preferably (10 to 20):1.

Method for Preparing the mRNA-Liposome Complex

In a second aspect of the present disclosure, the present disclosure provides a method for preparing the mRNA-liposome complex according to the first aspect. The method includes: mixing the liposome with the nucleic acid, to obtain the mRNA-liposome complex. The method according to the embodiment of the present disclosure can prepare the mRNA-liposome complexes in large scale, and has the advantages of simple preparation process, low device requirements, and high preparation efficiency.

In some optional embodiments of the present disclosure, the above method may further include at least one of the following technical features.

In some optional embodiments of the present disclosure, the liposome is obtained by: dissolve each of a core lipid, an auxiliary lipid, a steroid, and a PEG lipid with an organic solvent, and obtaining a core lipid solution, an auxiliary lipid solution, a steroid solution, and a PEG lipid solution; and mixing the core lipid solution, the auxiliary lipid solution, the steroid solution, and the PEG lipid solution in a first buffer, to obtain the liposome.

The organic solvent is an alcohol organic solvent. The alcohol organic solvent is selected from C₁ to C₄ alcohol organic solvent, such as C₁ to C₂ alcohol organic solvent. The alcohol organic solvent is selected from at least one of methanol, ethanol, propanol, and butanol, and preferably, the alcohol organic solvent is selected from methanol and ethanol, and more preferably, the alcohol organic solvent is ethanol, such as anhydrous ethanol.

In some optional embodiments of the present disclosure, the first buffer is selected from at least one of a sodium citrate buffer, an acetate buffer, and a sodium bicarbonate buffer.

In some optional embodiments of the present disclosure, after the core lipid solution, the auxiliary lipid solution, the steroid solution, and the PEG lipid solution are mixed in the buffer solution, the mixture is further subjected to a dialysis treatment.

In some optional embodiments of the present disclosure, the dialysis treatment is performed using a dialysis membrane having a molecular weight cut-off ranging from 50 kD to 500 kD, for example, 60 kD, 70 kD, 80 kD, 90 kD, 100 kD, 110 kD, 120 kD, 130 kD, 140 kD, 150 kD, 200 kD, 250 kD, 300 kD, 350 kD, 400 kD, or 450 kD.

In some optional embodiments of the present disclosure, before mixing the liposome with the nucleic acid, the mRNA is dissolved using a second buffer.

In some optional embodiments of the present disclosure, the second buffer is selected from at least one of a sodium citrate buffer, an acetate buffer, and a sodium bicarbonate buffer.

In some optional embodiments of the present disclosure, the second buffer solution may further include an ethanol aqueous solution with a volume percentage concentration ranging from 20% to 30%.

### Pharmaceutical Composition

In a third aspect of the present disclosure, the present disclosure provides a pharmaceutical composition. The pharmaceutical composition includes: the mRNA-liposome complex according to the first aspect or the mRNA-liposome complex prepared by the method according to the second aspect; and optionally, a pharmaceutically acceptable excipient or carrier. As mentioned above, the aforementioned mRNA-liposome complex has the advantages of low cytotoxicity, good biocompatibility, and strong delivery ability. Therefore, the pharmaceutical composition containing the aforementioned mRNA-liposome complex can deliver mRNA to the cells in the body, allow related proteins to be expressed in large quantities, thereby exerting the efficacy of treating the hyperuricemia-related diseases.

The pharmaceutical composition of the present disclosure may be administrated in any acceptable route. The pharmaceutical composition of the present disclosure may be formulated into solid, semi-solid, liquid, or gaseous forms, such as injections and lyophilized powders. The available methods for preparing these dosage forms are known or apparent to those skilled in the art. Typical administration routes of the pharmaceutical composition include, but are not limited to, oral, topical, transdermal, inhalation, parenteral, sublingual, buccal, rectal, vaginal, and intranasal routes. The term “parenteral” as used herein includes subcutaneous injections, intravenous injections, intramuscular injections, intradermal injections, intrasternal injection, or infusion techniques. The pharmaceutical compositions of the present disclosure are formulated, allowing the biologically active ingredients contained therein to be bioavailable upon administration of the composition to a patient.

### Uses

In a fourth aspect of the present disclosure, the present disclosure provides use of the mRNA-liposome complex according to the first aspect, the mRNA-liposome complex prepared by the method according to the second aspect, or the pharmaceutical composition according to the third aspect in the preparation of a medicament. The medicament has at least one of the following effects: introducing mRNA into cells, delivering mRNA mainly to the liver, increasing the expression or activity of related protein, and preventing and/or treating hyperuricemia-related diseases.

In some optional embodiments of the present disclosure, the related protein is selected from at least one of recombinant urate oxidase and urate oxidase.

In some optional embodiments of the present disclosure, the hyperuricemia-related diseases are selected from at least one of hyperuricemia, gout, gouty nephropathy, gouty vasculopathy, and gouty cardiomyopathy.

In a fifth aspect of the present disclosure, the present disclosure provides use of the mRNA-liposome complex according to the first aspect, the mRNA-liposome complex prepared by the method according to the second aspect, or the pharmaceutical composition according to the third aspect in at least one of introducing mRNA into cells, delivering mRNA mainly to the liver, increasing the expression or activity of related protein, and preventing and/or treating hyperuricemia-related diseases.

In some optional embodiments of the present disclosure, the related protein is selected from at least one of recombinant urate oxidase and urate oxidase.

In some optional embodiments of the present disclosure, the hyperuricemia-related diseases are selected from at least one of hyperuricemia, gout, gouty nephropathy, gouty vasculopathy, and gouty cardiomyopathy.

In a sixth aspect of the present disclosure, the present disclosure provides the mRNA-liposome complex according to the first aspect, the mRNA-liposome complex prepared by the method according to the second aspect, or the pharmaceutical composition according to the third aspect for use in at least one of introducing mRNA into cells, delivering mRNA mainly to the liver, increasing the expression or activity of related protein, and preventing and/or treating hyperuricemia-related diseases.

In some optional embodiments of the present disclosure, the related protein is selected from at least one of recombinant urate oxidase and urate oxidase.

In some optional embodiments of the present disclosure, the hyperuricemia-related diseases are selected from at least one of hyperuricemia, gout, gouty nephropathy, gouty vasculopathy, and gouty cardiomyopathy.

### Methods

In a seventh aspect of the present disclosure, the present disclosure provides a method for treating and/or preventing hyperuricemia-related diseases. The method includes administering a pharmaceutically acceptable amount of the mRNA-liposome complex according to the first aspect, the mRNA-liposome complex prepared by the method according to the second aspect, or the pharmaceutical composition according to the third aspect to a subject. As mentioned above, the aforementioned mRNA-liposome complex has the advantages of low cytotoxicity, good biocompatibility, and strong delivery ability. Therefore, the pharmaceutical composition containing the aforementioned mRNA-liposome complex can deliver mRNA to cells in the body and allow related proteins to be expressed in large quantities, thereby exerting the efficacy of treating the hyperuricemia-related diseases.

In some optional embodiments of the present disclosure, the hyperuricemia-related diseases are selected from at least one of hyperuricemia, gout, gouty nephropathy, gouty vasculopathy, and gouty cardiomyopathy.

The effective amount or acceptable amount of the mRNA-liposome complex or pharmaceutical composition of the present disclosure may vary depending on the administration route and the severity of the disease to be treated. The preferred effective amount may be selected and determined by those skilled in the art based on various factors (for example, through clinical trials). The factors include, but are not limited to: pharmacokinetic parameters of the biologically active ingredient (e.g., bioavailability, metabolism, half-life, etc.), severity of the disease to be treated of the subject, weight of the subject, immune status of the subject, route of administration, etc. For example, several divided doses may be administered daily, or the dose may be proportionally reduced as required by the exigencies of the therapeutic situation.

The mRNA-liposome complex or pharmaceutical composition of the present disclosure may be incorporated into medicaments suitable for parenteral administration (e.g., intravenous, subcutaneous, intraperitoneal, intramuscular). These medicaments may be prepared in various forms, such as liquid, semi-solid, and solid formulations, etc. The formulations of the medicaments include but are not limited to liquid solutions (e.g., injection solutions and infusion solutions) or lyophilized powders. The typical drug is the injection solution. The aforementioned mRNA-liposome complex or pharmaceutical composition may be administered by intravenous infusion or injection, intramuscular injection, or subcutaneous injection.

In some optional embodiments of the present disclosure, the administration route of the method is subcutaneous injection or intravenous injection.

In an eighth aspect of the present disclosure, the present disclosure provides a method for delivering mRNA mainly to liver cells. The method includes: administering a pharmaceutically acceptable amount of the mRNA-liposome complex according to the first aspect, the mRNA-liposome complex prepared by the method according to the second aspect, or the pharmaceutical composition according to the third aspect to a subject.

In some optional embodiments of the present disclosure, the administration route of the method is via a systemic administration route.

In some optional embodiments of the present disclosure, the cells are derived from mammals.

In some optional embodiments of the present disclosure, the mammal is selected from humans or mice.

In some optional embodiments of the present disclosure, the administration route of the method is subcutaneous injection or intravenous injection.

In a ninth aspect of the present disclosure, the present disclosure provides a method for increasing the expression or activity of urate oxidase or recombinant urate oxidase. The method includes: administering a pharmaceutically acceptable amount of the mRNA-liposome complex according to the first aspect, the mRNA-liposome complex prepared by the method according to the second aspect, or the pharmaceutical composition according to the third aspect to a subject.

In some optional embodiments of the present disclosure, the administration route of the method is via a systemic administration route.

In some optional embodiments of the present disclosure, the administration route of the method is subcutaneous injection or intravenous injection.

In a tenth aspect of the present disclosure, the present disclosure provides a method for treating and/or improving one or more hyperuricemia-related diseases in humans. The method includes: administering a therapeutically effective amount of the mRNA-liposome complex according to the first aspect or the pharmaceutical composition according to the second aspect to the human, where the mRNA in the mRNA-liposome complex encodes recombinant urate oxidase and/or urate oxidase.

The amino acid sequences or nucleotide sequences involved in the present disclosure are shown in the following table.

| Name | Amino acid sequence or nucleotide sequence | SEQ ID NO: |
|---|---|---|
| Murine UOX | MAHYHDNYGKNDEVEFVRTGYGKDMVKVLHIQRDGKYHSIKEVAT | 1 |
| protein | SVQLTLRSKKDYLHGDNSDIIPTDTIKNTVHVLAKLRGIRNIETFAMN | |
| | ICEHFLSSFNHVTRAHVYVEEVPWKRFEKNGIKHVHAFIHTPTGTHF | |
| | CEVEQMRNGPPVIHSGIKDLKVLKTTQSGFEGFLKDQFTTLPEVKDR | |
| | CFATQVYCKWRYQRRDVDFEAIWGAVRDIVLQKFAGPYDKGEYSPS | |
| | VQKTLYDIQVLSLSQLPEIEDMEISLPNIHYFNIDMSKMGLINKEEVL | |
| | LPLDNPYGKITGTVKRKLPSRL | |
| Rhesus monkey | MADYHNNYKKNDELEFVRTGYGKDMVKVLHIQRDGKYHSIKEVAT | 2 |
| UOX protein | SVQLTLSSKKDYVHGDNSDIIPTDTIKNTVHVLAKLKGIKSIEAFGVN | |
| | ICEYFLSSFNHVIRAQVYVEEIPWKRLEKNGVKHVHAFIHTPTGTHF | |
| | CEVEQLRSGPPV | |
| | IHSGIKDLKVLKTTQSGFEGFIKDQFTTLPEVKDRCFATQVYCKWRY | |
| | HQCRDVDFEATWGTIRDLVLEKFAGPYDKGEYSPSVQKTLYDIQVLS | |
| | LSRVPEIEDMEISLPNIHYFNIDMSKMGLINKEEVLLPLDNPYGKITGT | |
| | VKRKLSSRL | |
| Recombinant | MSAVKAARYGKDNVRVYKVHKDEKTGVQTVYEMTVCVLLEGEIET | 3 |
| urate oxidase | SYTKADNSVIVATDSIKNTIYITAKQNPVTPPELFGSILGTHFIEKYNHI | |
| | HAAHVNIVCHRWTRMDIDGKPHPHSFIRDSEEKRNVQVDVVEGKGI | |
| | DIKSSLSGLTVLKSTNSQFWGFLRDEYTTLKETWDRILSTDVDATWQ | |
| | WKNFSGLQEVRSHVPKFDATWATAREVTLKTFAEDNSASVQATMYK | |
| | MAEQILARQQLIETVEYSLPNKHYFEIDLSWHKGLQNTGKNAEVFA | |
| | PQSDPNGLIKCTVGRSSLKSKL | |
| Uox1 | MAHYRNTYKKNDEVEFVRTGYGKDMIKVLHIQRDGKYHSIKEVAT | 4 |
| | TVQLTLSSKKDYLHGDNSDVIPTDTIKNTVNVLAKFKGIKSIETFAVTI | |
| | CEHFLSSFKHVIRAQVYVEEVPWKRFEKNGVKHVHAFIYTPTGTHF | |
| | CEVEQIRNGPPVIHSGIKDLKVLKTTQSGFEGFIKDQFTTLPEVKDRC | |
| | FATQVYCKWRYHQGRDVDFEATWDTVRSIVLQKFAGPYDKGEYSPS | |
| | VQKTLYDIQVLTLGQVPEIEDMEISLPNIHYLNIDMSKMGLINKEEVL | |
| | LPLDNPYGKITGTVKRKLSSRL | |
| Uox2 | MAHYHNNYKKNDEVEFVRTGYGKEMVKVLHIQRDGKYHSIKEVAT | 5 |
| | SVQLTLSSKKDYLHGDNSDIIPTDTIKNTVHVLAKFKEIKSIEAFGVNI | |
| | CEHFLSSFNHVIRAQVYMEEIPWKHLGKNGVKHVHAFIHTPTGTHF | |
| | CEVEQLRSGPQVIHSGIKDLKVLKTTQSGFEGFIKDQFTTLPEVKDRC | |
| | FATQVYCKWRYHQCRDVDFKATWDTIRDLVMEKSAGPYDKGEYLT | |
| | SVQKTLCDIQVLSLSRVPAIEDMEISLPNIHYFNIDMSKMGLINKEEV | |
| | RGLAAIRQSIWKNYWYSQEEVVFK | |
| Uox3 | MADYHNNYKKNDELEFVRTGYGKDMVKVLHIQRDGKYHSIKEVAT | 6 |
| | SVQLTLSSKKDYLHGDNSDIIPTDTIKNTVHVLAKFKGIKSIEAFGVNI | |
| | CEYFLSSFNHVIRAQVYVEEIPWKRLEKNGVKHVHAFIHTPTGTHFC | |
| | EVEQLRSGPPVIHSGIKDLKVLKTTQSGFEGFIKDQFTTLPEVKDRCF | |
| | ATQVYCKWRYHQCRDVDFEATWGTIRDLVLEKFAGPYDKGEYSPSV | |
| | QKTLYDIQVLSLSRVPEIEDMEISLPNIHYFNIDMSKMGLINKEEVLLP | |
| | LDNPYGKITGTVKRKLSSRL | |
| Murine | ATGGCCCATTACCATGACAACTACGGAAAGAATGATGAAGTGGAA | 7 |
| UoxmRNA | TTTGTCCGAACTGGCTATGGGAAAGACATGGTCAAAGTTCTCCAT | |
| | ATTCAGAGAGATGGAAAATACCACAGCATCAAAGAGGTGGCAAC | |
| | TTCGGTCCAGTTGACTCTGAGGTCCAAGAAGGATTACCTACACGG | |
| | TGATAATTCCGACATCATCCCTACAGACACCATCAAGAACACTGTG | |
| | CACGTCCTGGCGAAGCTCAGAGGGATCAGAAACATCGAGACCTT | |
| | TGCAATGAACATCTGTGAGCACTTCCTCTCTTCTTTTAACCATGTC | |
| | ACTCGAGCCCACGTCTACGTGGAAGAGGTCCCCTGGAAACGATTT | |
| | GAAAAGAATGGGATCAAACACGTCCATGCATTCATCCACACCCCG | |
| | ACGGGAACGCACTTCTGTGAGGTGGAGCAGATGAGAAACGGACC | |
| | TCCCGTCATTCACTCTGGAATCAAAGACCTCAAGGTCTTGAAAAC | |
| | AACCCAGTCTGGGTTTGAAGGGTTCCTCAAGGACCAGTTCACTAC | |
| | CCTCCCTGAGGTGAAGGACCGATGCTTTGCCACTCAAGTGTACTG | |
| | CAAGTGGCGCTACCAGAGACGGGACGTGGACTTCGAGGCTATCT | |
| | GGGGCGCTGTCCGGGACATTGTCCTGCAGAAATTTGCTGGGCCCT | |
| | ATGACAAAGGTGAATACTCACCTTCCGTGCAGAAGACCCTCTATG | |
| | ATATACAAGTGCTGTCCCTGAGCCAGCTTCCTGAGATAGAAGACA | |
| | TGGAAATCAGCCTTCCGAACATTCACTACTTTAACATCGACATGTC | |
| | CAAAATGGGACTGATCAACAAGGAAGAGGTTTTGCTGCCTCTCGA | |
| | CAATCCCTACGGCAAAATAACAGGGACAGTCAAGAGGAAGCTGC | |
| | CTTCCAGGCTGTGA | |
| Rhesus monkey | ATGGCCGACTACCATAACAACTATAAAAAGAATGATGAGTTGGAG | 8 |
| UoxmRNA | TTTGTCCGAACTGGCTATGGGAAGGATATGGTAAAAGTTCTCCATA | |
| | TTCAGCGAGATGGAAAATATCACAGCATTAAAGAGGTGGCAACTT | |
| | CAGTGCAACTTACTCTGAGTTCCAAAAAAGATTACGTGCATGGAG | |
| | ATAATTCAGATATCATCCCTACAGACACCATCAAGAACACAGTTCA | |
| | TGTCTTGGCAAAGTTGAAGGGAATCAAAAGCATAGAAGCCTTTGG | |
| | TGTGAATATTTGTGAGTATTTTCTTTCTTCTTTTAACCATGTAATCC | |
| | GAGCTCAAGTCTACGTGGAAGAAATCCCTTGGAAGCGTCTTGAA | |
| | AAGAATGGAGTTAAGCATGTCCATGCATTTATTCACACTCCCACTG | |
| | GAACACACTTCTGTGAAGTTGAACAACTGAGAAGTGGACCCCCC | |
| | GTCATTCATTCTGGAATCAAAGACCTCAAGGTCTTGAAAACAACA | |
| | CAGTCTGGATTTGAAGGTTTCATCAAGGACCAGTTCACCACCCTC | |
| | CCTGAGGTGAAGGACCGATGCTTTGCCACCCAAGTGTACTGCAAG | |
| | TGGCGCTACCACCAGTGCAGGGATGTGGACTTCGAGGCTACCTGG | |
| | GGCACCATTCGGGACCTTGTCCTGGAGAAATTTGCTGGGCCCTAT | |
| | GACAAAGGCGAGTACTCGCCCTCTGTGCAGAAGACCCTCTATGAT | |
| | ATACAGGTGCTCTCCCTGAGCCGAGTTCCTGAGATAGAAGATATG | |
| | GAAATCAGCCTGCCAAACATTCACTACTTCAATATAGACATGTCCA | |
| | AAATGGGTCTGATCAACAAGGAAGAGGTCTTGCTGCCATTAGACA | |
| | ATCCATATGGAAAAATTACTGGTACAGTCAAGAGGAAGTTGTCTT | |
| | CAAGACTGTGA | |
| Recombinant | ATGAGCGCCGTGAAAGCGGCCCGTTATGGCAAAGATAATGTCCGC | 9 |
| urate oxidase | GTGTACAAAGTTCATAAAGACGAAAAAACCGGCGTTCAGACGGT | |
| mRNA | CTATGAAATGACCGTTTGCGTCCTGCTGGAAGGTGAAATTGAAAC | |
| | CTCATACACGAAAGCGGATAACTCGGTGATTGTTGCCACCGACAG | |
| | CATCAAAAACACCATTTATATCACGGCGAAACAGAATCCGGTGAC | |
| | CCCGCCGGAACTGTTTGGCAGTATTCTGGGTACGCACTTCATCGA | |
| | AAAATACAACCATATTCACGCAGCTCATGTGAATATCGTTTGTCAC | |
| | CGTTGGACCCGCATGGATATTGACGGCAAACCGCATCCGCACAGT | |
| | TTTATCCGTGATTCCGAAGAAAAACGCAACGTCCAGGTGGATGTG | |
| | GTTGAAGGCAAAGGTATTGACATCAAAAGCTCTCTGTCTGGCCTG | |
| | ACCGTGCTGAAAAGCACCAACAGCCAATTTTGGGGTTTCCTGCGT | |
| | GATGAATATACCACGCTGAAAGAAACCTGGGACCGCATTCTGAGC | |
| | ACCGATGTTGACGCGACGTGGCAGTGGAAAAATTTTTCTGGTCTG | |
| | CAAGAAGTCCGTAGTCATGTGCCGAAATTCGATGCAACCTGGGCG | |
| | ACGGCCCGCGAAGTTACCCTGAAAACGTTCGCTGAAGACAACTC | |
| | AGCATCGGTCCAGGCTACCATGTACAAAATGGCAGAACAAATCCT | |
| | GGCTCGTCAGCAACTGATTGAAACGGTGGAATATTCCCTGCCGAA | |
| | CAAACATTACTTCGAAATTGATCTGTCATGGCACAAAGGCCTGCA | |
| | GAACACCGGTAAAAATGCGGAAGTCTTCGCCCCGCAAAGCGATC | |
| | CGAACGGCCTGATCAAATGCACCGTTGGTCGCAGTTCCCTGAAAA | |
| | GCAAACTGTGA | |
| Uox1 mRNA | ATGGCTCATTACCGTAATACGTACAAAAAGAATGATGAGGTAGAG | 10 |
| | TTTGTCCGAACTGGCTATGGGAAGGATATGATAAAAGTTCTCCATA | |
| | TTCAGCGAGATGGAAAATATCACAGCATTAAAGAGGTGGCAACTA | |
| | CAGTGCAACTGACTTTGAGCTCCAAAAAAGATTACCTGCATGGAG | |
| | ACAATTCAGATGTCATCCCTACAGACACCATCAAGAACACAGTTA | |
| | ATGTCCTGGCGAAGTTCAAAGGCATCAAAAGCATAGAAACTTTTG | |
| | CTGTGACTATCTGTGAGCATTTCCTTTCTTCCTTCAAGCATGTCATC | |
| | AGAGCTCAAGTCTATGTGGAAGAAGTTCCTTGGAAGCGTTTTGAA | |
| | AAGAATGGAGTTAAGCATGTCCATGCATTTATTTATACTCCTACTGG | |
| | AACGCACTTCTGTGAGGTTGAACAGATAAGGAATGGACCTCCAGT | |
| | CATTCATTCTGGAATCAAAGACCTAAAAGTCTTGAAAACAACCCA | |
| | GTCTGGCTTTGAAGGATTCATCAAGGACCAGTTCACCACCCTCCC | |
| | TGAGGTGAAGGACCGGTGCTTTGCCACCCAAGTGTACTGCAAATG | |
| | GCGCTACCACCAGGGCAGAGATGTGGACTTTGAGGCCACCTGGG | |
| | ACACTGTTAGGAGCATTGTCCTGCAGAAATTTGCTGGGCCCTATG | |
| | ACAAAGGCGAGTACTCGCCCTCTGTCCAGAAGACACTCTATGACA | |
| | TCCAGGTGCTCACCCTGGGCCAGGTTCCTGAGATAGAAGATATGG | |
| | AAATCAGCCTGCCAAATATTCACTACTTAAACATAGACATGTCCAA | |
| | AATGGGACTGATCAACAAGGAAGAGGTCTTGCTACCTTTAGACAA | |
| | TCCATATGGCAAGATTACTGGTACAGTCAAGAGGAAGCTGTCTTC | |
| | AAGGCTGTGA | |
| Uox2 mRNA | ATGGCCCACTACCATAACAACTATAAAAAGAATGATGAGGTGGAG | 11 |
| | TTTGTCCGAACTGGCTATGGGAAGGAAATGGTAAAAGTTCTCCAT | |
| | ATTCAGCGAGATGGAAAATATCACAGCATTAAAGAGGTGGCAACT | |
| | TCAGTGCAACTTACTCTAAGTTCCAAAAAAGATTACCTGCATGGA | |
| | GATAATTCAGACATCATCCCTACAGACACCATCAAGAACACAGTT | |
| | CATGTCTTGGCAAAGTTTAAAGAAATCAAAAGCATAGAAGCCTTT | |
| | GGTGTGAATATTTGTGAGCATTTTCTTTCTTCTTTTAACCATGTAAT | |
| | CCGAGCTCAAGTCTACATGGAAGAAATCCCTTGGAAGCATCTTGG | |
| | AAAGAATGGAGTTAAGCATGTCCATGCATTTATTCACACTCCCACT | |
| | GGAACACACTTCTGTGAAGTTGAACAGCTGAGAAGTGGACCCCA | |
| | AGTCATTCATTCTGGAATCAAAGACCTCAAGGTCTTGAAAACAAC | |
| | ACAGTCTGGATTTGAAGGTTTCATCAAGGACCAGTTCACTACCCT | |
| | CCCTGAGGTGAAGGACCGATGCTTTGCCACCCAAGTGTACTGCAA | |
| | GTGGCGCTACCACCAGTGCAGGGATGTGGACTTCAAGGCTACCTG | |
| | GGACACCATTCGGGACCTTGTCATGGAGAAATCTGCTGGGCCCTA | |
| | TGACAAAGGTGAATACTTGACCTCTGTGCAGAAGACCCTCTGTGA | |
| | TATCCAGGTGCTCTCCCTGAGCCGAGTTCCTGCGATAGAAGATATG | |
| | GAAATCAGCCTGCCAAACATTCACTACTTCAACATAGACATGTCC | |
| | AAAATGGGTCTGATCAACAAGGAAGAGGTAAGAGGTCTTGCTGC | |
| | CATTAGACAATCCATATGGAAAAATTACTGGTACAGTCAAGAGGA | |
| | AGTTGTCTTCAAGTGA | |
| Uox3 mRNA | ATGGCCGACTACCATAACAACTATAAAAAGAATGATGAGTTGGAG | 12 |
| | TTTGTCCGAACTGGCTATGGGAAGGATATGGTAAAAGTTCTCCATA | |
| | TTCAGCGAGATGGAAAATATCACAGCATTAAAGAGGTGGCAACTT | |
| | CAGTGCAACTTACTCTGAGTTCCAAAAAAGATTACCTGCATGGAG | |
| | ATAATTCAGATATCATCCCTACAGACACCATCAAGAACACAGTTCA | |
| | TGTCTTGGCAAAGTTTAAGGGAATCAAAAGCATAGAAGCCTTTGG | |
| | TGTGAATATTTGTGAGTATTTTCTTTCTTCTTTTAACCATGTAATCC | |
| | GAGCTCAAGTCTACGTGGAAGAAATCCCTTGGAAGCGTCTTGAA | |
| | AAGAATGGAGTTAAGCATGTCCATGCATTTATTCACACTCCCACTG | |
| | GAACACACTTCTGTGAAGTTGAACAACTGAGAAGTGGACCCCCC | |
| | GTCATTCATTCTGGAATCAAAGACCTCAAGGTCTTGAAAACAACA | |
| | CAGTCTGGATTTGAAGGTTTCATCAAGGACCAGTTCACCACCCTC | |
| | CCTGAGGTGAAGGACCGATGCTTTGCCACCCAAGTGTACTGCAAG | |
| | TGGCGCTACCACCAGTGCAGGGATGTGGACTTCGAGGCTACCTGG | |
| | GGCACCATTCGGGACCTTGTCCTGGAGAAATTTGCTGGGCCCTAT | |
| | GACAAAGGCGAGTACTCGCCCTCTGTGCAGAAGACCCTCTATGAT | |
| | ATACAGGTGCTCTCCCTGAGCCGAGTTCCTGAGATAGAAGATATG | |
| | GAAATCAGCCTGCCAAACATTCACTACTTCAATATAGACATGTCCA | |
| | AAATGGGTCTGATCAACAAGGAAGAGGTCTTGCTGCCATTAGACA | |
| | ATCCATATGGAAAAATTACTGGTACAGTCAAGAGGAAGTTGTCTT | |
| | CAAGACTGTGA | |
| 5'UTR | TCAAGCTTTTGGACCCTCGTACAGAAGCTAATACGACTCACTATAG | 13 |
| | GGAAATAAGAGAGAAAAGAAGAGTAAGAAGAAATATAAGAGCCA | |
| | CCATGGAGACAGACACACTCCTGCTATGGGTACTGCTGCTCTGGG | |
| | TTCCAGGTTCCACTGGTGAC | |
| 3'UTR | TGATAATAGGCTGGAGCCTCGGTGGCCATGCTTTCTTGCCCCTTGG | 14 |
| | GCCTCCCCCCAGCCCCTCCTCCCCTTCCTGCACCCGTACCCCCGTG | |
| | GTCTTTGAATAAAAGTCTGAGTGGGCGGC | |

The solutions of the present disclosure will be explained below with reference to examples. Those skilled in the art will appreciate that the following examples are only used to illustrate the present disclosure and should not be considered to limit the scope of the present disclosure. Techniques or conditions that are not specified in the examples shall be performed in accordance with the techniques or conditions described in the literatures in the art or in accordance with the product instruction. Reagents or instruments without indicating the manufacturer are all common products that are commercially available.

Unless otherwise defined, all the technical and scientific terms used herein have the same meaning as commonly understood by those skilled in the art to which the present disclosure belongs. Although any methods and materials similar or equivalent to those described herein can be used in the practice or testing of the formulations or unit dosages herein, some methods and materials are now described. Unless otherwise stated, the techniques employed or considered herein are standard methods. The materials, methods, and examples are illustrative only and not limiting.

### Example 1: Construction of Uox Plasmid and Preparation of mUox

1. The mRNA sequence of murine Uox was obtained from the NCBI database (Gene ID: 22262), CDS region of the sequence was selected, and the specific nucleotide sequence was set forth in SEQ ID No: 7 (the amino acid sequence of the protein encoded by the mRNA sequence was set forth in SEQ ID NO: 1). Subsequently, 3′UTR region (nucleotide sequence thereof was set forth in SEQ ID NO: 14) and 5′UTR region (nucleotide sequence thereof was set forth in SEQ ID NO: 13) were sequentially added to the optimized CDS region, to obtain the optimized mUox sequence. The optimized mUox sequence was inserted into a pcDNA3.1 vector to obtain a plasmid stably expressing the Uox gene. The plasmid was transformed into competent Escherichia coli (E. coli) cells. The suspension of the competent cells was taken by dipping and streaked onto the prepared LB medium plate, and the cells were cultured at 37° C. for 12 hours to 16 hours. Subsequently, a single clone colony was picked for amplification, to obtain a plasmid bacterial solution stably expressing the Uox gene, which was packaged and stored at −80° C. The plasmid was extracted, linearized, and transcribed in vitro to obtain the mUox.

2. The above method was used to prepare Uox1 mRNA, Uox2 mRNA, and Uox3 mRNA. The nucleotide sequence of the CDS region of Uox1 mRNA was set forth in SEQ ID NO: 10, and the amino acid sequence of the protein encoded by Uox1 mRNA was set forth in SEQ ID NO: 4. The nucleotide sequence of the CDS region of Uox2 mRNA was set forth in SEQ ID NO: 11, and the amino acid sequence of the protein encoded by Uox2 mRNA was set forth in SEQ ID NO: 5. The nucleotide sequence of the CDS region of Uox3 mRNA was set forth in SEQ ID No: 12, and the amino acid sequence of the protein encoded by Uox3 mRNA was set forth in SEQ ID No: 6.

### Example 2: Preparation of Lipid Nanoparticles

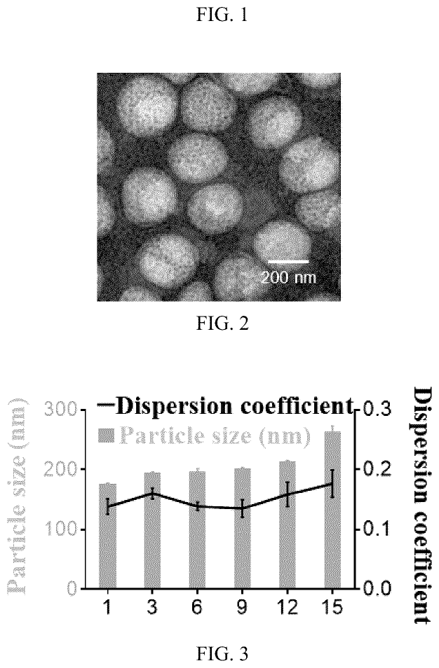

1) A1-D1-5 (which may be prepared by conventional methods in the art and be the same as A1-D1-5 in other examples, and the specific chemical structural formula was shown in FIG. 1), cholesterol, DOPE, and DMG-PEG2000 were taken and dissolved in anhydrous ethanol, to form a 2 mg/mL organic phase solution. A molar percentage of A1-D1-5 was 35.1%, a ratio of DOPE was 11.7%, a ratio of cholesterol was 52.6%, and a ratio of DMG-PEG2000 was 0.6%.

2) The organic phase solution was rapidly injected into a 3-fold volume of 50 mM sodium citrate buffer solution (pH=4.0) while being stirred at a high speed to form iLAND.

3) The mRNAs obtained in Example 1 (mUox, Uox1 mRNA, Uox2 mRNA, and Uox3 mRNA) were mixed with iLAND in equal volumes (a mass ratio of iLAND to mRNA was 15:1) and incubated at 50° C. for 10 minutes.

4) The incubated solution was transferred to a 100 kD dialysis bag and dialyzed in 1×PBS for 2 hours, to obtain mUox@iLAND, Uox1@iLAND, Uox2@iLAND, and Uox3@iLAND of the present disclosure.

### Example 3: Characterization and Stability Test of Lipid Nanoparticles

10 μL of each of mUox@iLAND, Uox1@iLAND, Uox2@iLAND, and Uox3@iLAND obtained in Example 2 were taken, dropped onto a copper grid, and allowed to stand for 10 minutes. The excess liquid on the surface was washed off, and staining was performed with 2% uranyl acetate three times for 1 min each time, followed by washing three times with PBS. After the copper grid was fully dried, a transmission electron microscope (HT7700) was used to observe the size and morphology of mUox@iLAND. The prepared mUox@iLAND solution was placed in a Malvern particle size analyzer, and its particle size and potential were detected by the principle of dynamic light scattering. The particle size was measured on the 1-st, 3-rd, 6-th, 9-th, 12-th, and 15-th day after the preparation to monitor the stability. The results of mUox@iLAND, Uox1@iLAND, Uox2@iLAND, and Uox3@iLAND were similar, and the result of mUox@iLAND was shown in FIG. 2, FIG. 3, and Table 1.

As shown in the transmission electron microscopy results in FIG. 2, mUox@iLAND was spherical and has a particle size of about 200 nm. As shown in Table 1, the hydrated particle size of mUox@iLAND was consistent with the electron microscopy results, which was about 170 nm; the PDI was less than 0.2, indicating that it has good uniformity; and the potential of mUox@iLAND was about 0 V, which was suitable for in vivo experimental analysis as a therapeutic drug. As shown in FIG. 3, mUox@iLAND remained relatively stable within 2 weeks.

TABLE 1
Hydrated particle size of mUox@iLAND

| Parameter | Result |
|---|---|
| Particle size (nm) | 169.1 ± 1.643 |
| Dispersion coefficient | 0.148 ± 0.005 |
| Potential (mV) | −1.22 ± 0.188 |

### Example 4: Determination of pKa of Lipid Nanoparticles (iLAND)

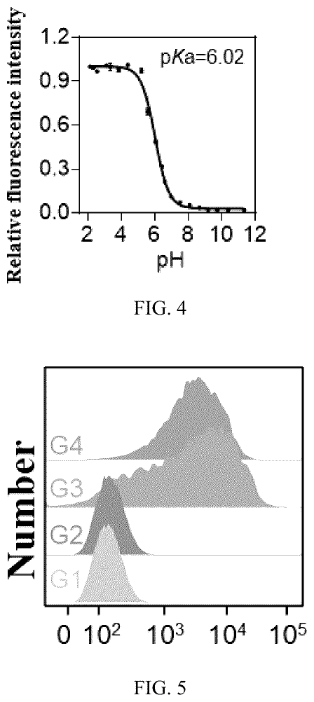

A series of buffer solutions (150 mM NaCl, 10 mM boric acid solution, 10 mM phosphoric acid solution, and 10 mM citric acid solution) with pH values ranging from 3 to 10 were first prepared. Blank lipid nanoparticles were prepared (the preparation method thereof was described in Example 2, except that no mRNA was added) and diluted in the respective solutions. 2-(p-toluidinyl)-6-naphthalenesulfonic acid (TNS) was dissolved in dimethyl sulfoxide (DMSO) to prepare a TNS solution with a TNS concentration of 300 μM, and 2 μL of the TNS solution was mixed with 100 μL of lipid nanoparticles. The fluorescence value was measured under the conditions of excitation light at 325 nm and emission light at 435 nm. FIG. 4 shows a curve of the fluorescence value and the pH value, and the results indicates that the pKa of the lipid nanoparticle iLAND was 6.02.

### Example 5: Cell Entry Effect of Lipid Nanoparticles

1. The lipid nanoparticles were prepared according to the method of Example 2, except that Cy5 fluorescently labeled mRNA (also known as Cy5 Nucleic acid or nucleic acid molecule) was used to obtain fluorescently labeled lipid nanoparticles (also known as Cy5 Nucleic acid@iLAND).

2. Hepa1-6 cells (purchased from CAS cell bank) were digested with trypsin and resuspended in DMEM complete medium containing 10% fetal bovine serum to adjust the cell density to about 1×10⁵ cells/mL. 1 mL of cell suspension was added to each well of the 12-well plate, and the cells were continued to culture in a cell culture incubator at 37° C. and 5% CO₂ for 24 hours to allow the cells to fully adhere to the wall. The lipid nanoparticles containing 0.8 μg Cy5 Nucleic acid prepared in step 1 were added to the 12-well plate, and flow/laser confocal analysis was performed after transfection for 24 hours.

Free Cy5 Nucleic acid was used as a negative control. 0.8 μg of Cy5 Nucleic acid was directly added into the well, gently shaken, and flow/laser confocal analysis was performed after transfection for 24 hours.

In addition, the commercial transfection reagent, Lipofectamine2000, was used as a positive control. Specifically, 2.4 μL of Lipofectamine2000 and 0.8 μg of Cy5 Nucleic acid were each added to 100 μL of Opti-MEM low serum medium for incubation. After 5 minutes, these two solutions were mixed and incubated for 15 minutes. The medium of cells in the well to be transfected with Lipofectamine2000 was changed in advance, the DMEM complete medium was replaced with Opti-MEM, and the incubated Cy5 Nucleic acid@Lipofectamine2000 solution was added to the wells and shaken gently. After transfection for four hours, 1 mL of DMEM complete medium was added, and the cells were collected after further culture for 20 hours for flow/laser confocal analysis.

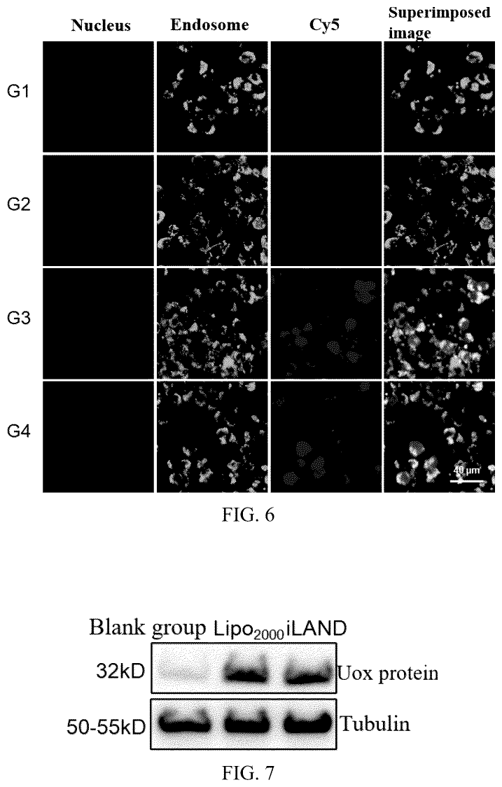

For the laser confocal assay, the cells of all experimental groups were stained for nuclei (Hoechst33342) and endosomes (Lysotracker Green), and the cell entry effect and endosome escape effect of Cy5 Nucleic acid@iLAND were observed under a laser confocal microscope. The results were shown in FIG. 5 and FIG. 6.

The flow confocal analysis results were shown in FIG. 5. The lipid nanoparticles Cy5 Nucleic acid@iLAND can mediate a good cell entry effect (G3), which is basically equivalent to the commercial transfection reagent Lipofectamine2000 (G4). However, Free Cy5 Nucleic acid cannot effectively enter cells due to the lack of a delivery vector (G2).

The laser confocal analysis results were shown in FIG. 6. The lipid nanoparticles Cy5 Nucleic acid@iLAND can mediate a good cell entry effect (G3), while Free Cy5 Nucleic acid cannot enter the cells (G2). The laser confocal results were consistent with the flow confocal results.

### Example 6: Expression of Lipid Nanoparticles at a Cellular Level

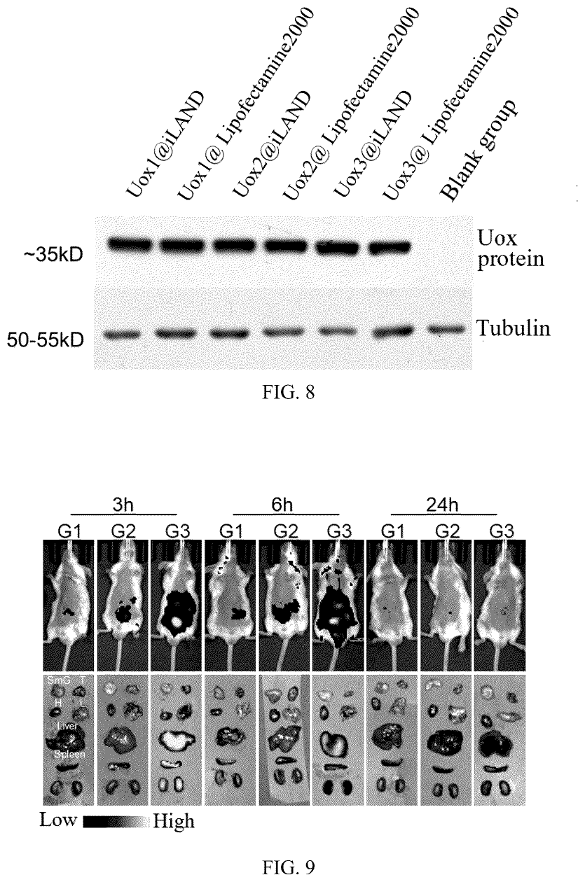

1. mUox@iLAND, Uox1@iLAND, Uox2@iLAND, and Uox3@iLAND (all were lipid nanoparticles without fluorescence labeling, and the specific preparation methods thereof were described in Example 2) were each transfected into Hepa1-6 cells, and mUox@Lipofectamine2000 was used as a positive control. After 24 hours of the transfection, the cells were collected and lysed. Total cell protein was extracted and the protein was quantified using a BCA protein quantification kit (Kangwei Century, CW0014S). The expression of Uox protein in the cells was detected by Western blotting. The results were shown in FIG. 7 and FIG. 8.

The results in FIG. 7 indicate that both the mUox@Lipofectamine2000 group and the mUox@iLAND group expressed the Uox protein, substantiating that the mUox sequence was successfully constructed and could achieve the correct expression of the Uox protein.

The results in FIG. 8 indicate that: both the Uox1@iLAND group and the Uox1@Lipofectamine2000 group can correctly express the Uox1 protein; both the Uox2@iLAND group and the Uox2@Lipofectamine2000 group can correctly express the Uox2 protein; and both the Uox3@iLAND group and the Uox3@Lipofectamine2000 group can correctly express the Uox3 protein. Therefore, the results reveal that the expression sequences designed in this example (Uox1 mRNA, Uox2 mRNA, and Uox3 mRNA) can achieve the correct expression of the novel Uox proteins (Uox1, Uox2, and Uox3).

### Example 7: Distribution and Expression of Lipid Nanoparticles (mRNA@iLAND) at an Animal Level

C57BL/6 mice aged 6 to 8 weeks were purchased from SPF (BEIJING) BIOTECHNOLOGY CO., LTD. The mice were randomly divided into three groups and respectively treated by injecting, through the tail vein, with PBS, Cy5 Nucleic acid@Lipofectamine2000 prepared in Example 5, and Cy5 Nucleic acid@iLAND prepared in Example 5. The animals were imaged with fluorescence and bioluminescence at 3, 6, and 24 hours after the injection to monitor the distribution and expression of mRNA. The mice were killed and dissected at different time points, and the submandibular gland, thymus, heart, liver, spleen, lung, and kidney were taken for imaging to more clearly observe the distribution and expression of mRNA in each organ. The liver tissues dissected at 6 hours and 24 hours were embedded in OCT gel, and then the embedded tissues were frozen and sectioned. The sections were stained with DAPI and FITC-labeled phalloidin in turn, and then fluorescence observation was performed using a laser confocal microscope. The results were shown in FIG. 9 to FIG. 13.

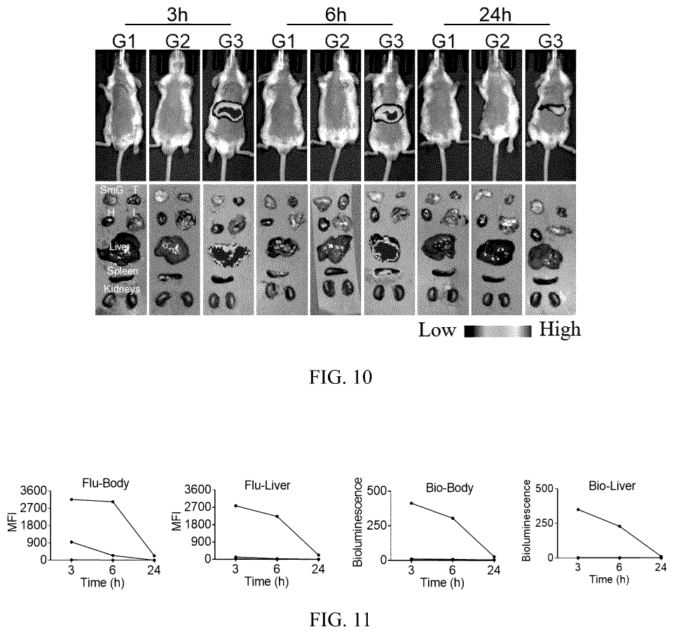

The in vivo distribution and expression results of the drug were shown in FIG. 9 and FIG. 10. The results indicate that: through intravenous administration, Cy5 Nucleic acid@Lipofectamine2000 (G2) was metabolized very quickly in the body, lasting for 3 to 6 hours (FIG. 9), and cannot achieve the function of in vivo protein expression (FIG. 10); while Cy5 Nucleic acid@iLAND (G3) may achieve long-term circulation of drug in vivo and may achieve efficient expression of the drug in the liver.

The quantitative results analysis in FIG. 11 reveal that the Cy5 Nucleic acid@iLAND group exhibited long-lasting pharmacokinetic distribution ability and protein expression level in the liver.

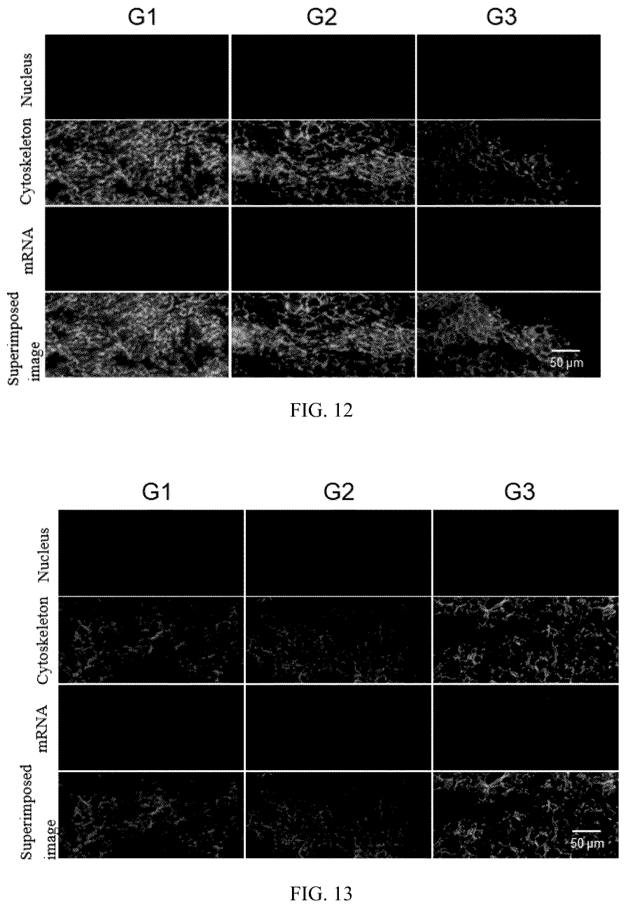

The frozen section results in FIG. 12 and FIG. 13 reveal that the fluorescently labeled nucleic acid molecules can be effectively enriched in the liver tissue, reaching a peak at 6 hours and being able to maintain for about 24 hours.

### Example 8: Therapeutic Effect of Lipid Nanoparticles (mUox@iLAND) in Model Animals

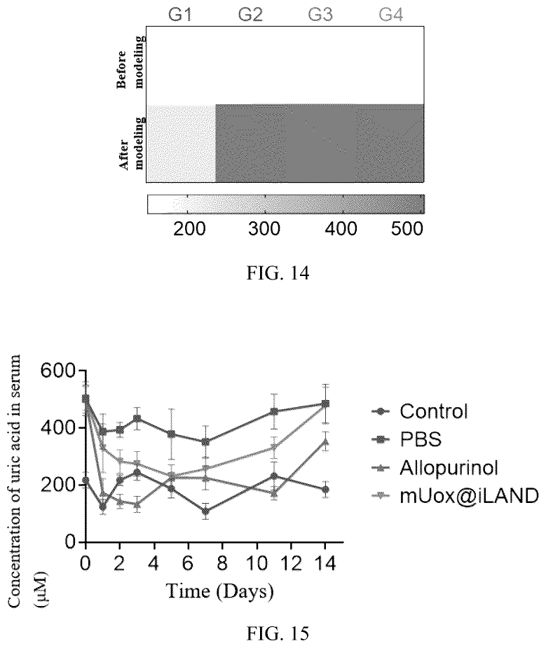

Female C57BL/6 mice aged 6 to 8 weeks were purchased from SPF (BEIJING) BIOTECHNOLOGY CO., LTD. The mice were randomly divided into 4 groups and subjected to different treatments, i.e., one group was fed normally, and the other three groups were constructed as models of hyperuricemia. For model mice, uric acid inhibitors were subcutaneously injected on Day −14. On Day 0, blood of all the mice was collected by orbital sampling, and serum was separated to detect uric acid levels. After confirming that the models of mice were successfully modeled, the mice were administrated with drug. The mice that were not subjected to the model construction were raised normally and administrated through tail vein injection with 200 μL of PBS (Control group, G1). The mice were subject to the model construction, and on 14 days after the model construction, the mice were administrated through tail vein injection with 200 μL of PBS (PBS group, G2). The mice were subject to the model construction, and on 14 days after the model construction, a certain amount of Allopurinol (which is a first-line clinical drug for anti-hyperuricemia, used as an experimental positive control group) was added to the drinking water, and the mice were allowed to drink the drinking water freely every day (Allopurinol group, G3). The mice were subject to the model construction, and on 14 days after the model construction, the mice were administrated through tail vein injection with 200 μL of mUox@iLAND prepared in Example 2 (mUox@iLAND group, G4), which was only administered once. The drugs were administered on the first day. Except for the Allopurinol group, which was administered daily, the other three groups were administered once during the whole course. At different time points after the drug administration, blood of the mice was collected by orbital sampling, and the serum was separated and analyzed for uric acid and other biochemical indices. The changes in weight of the mice were recorded throughout the model construction and drug administration. The results were shown in FIG. 14 to FIG. 17.

The results of model construction were shown in FIG. 14. Through the 14 days of model construction, the uric acid level in the model mice was significantly increased (from 100 μM to 400 μM), indicating that the hyperuricemia mouse model was successfully constructed.

The anti-hyperuricemia treatment results were shown in FIG. 15. Compared with the PBS group, the mice in the Allopurinol group and the mUox@iLAND group were able to maintain a certain level of uric acid. However, the drug was required to be administrated to the mice in the Allopurinol group every day, while the drug was administrated only once to the mice in the mUox@iLAND group. The results indicate that lipid nanoparticles mUox@iLAND can efficiently treat hyperuricemia. Compared with Allopurinol, which is a first-line clinical drug and required to be taken daily, uric acid can be maintained at normal levels for a long period of time by administrating mUox@iLAND only once, which exhibited more significant advantages.

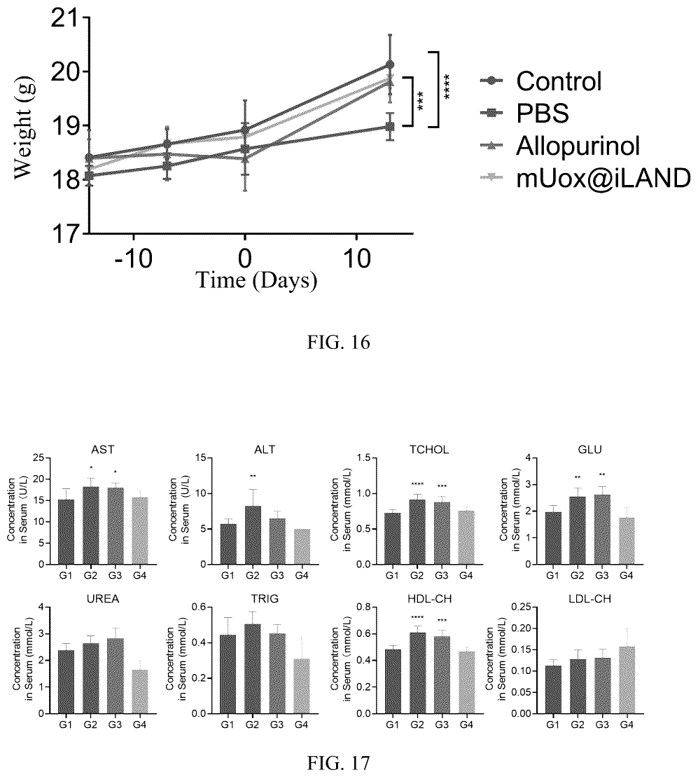

The changes in weight of mice in each group were shown in FIG. 16. The weight of mice in the mUox@iLAND group was similar to that in the Control group, indicating that mUox@iLAND has good safety.

The serum biochemical indicators in FIG. 17 reveal that mUox@iLAND showed no obvious toxic side effects, indicating that the safety of mUox@iLAND is superior over that of the clinical first-line drug Allopurinol.

### Example 9: Therapeutic Effect of Lipid Nanoparticles in Model Animals

Example 9 differed from Example 7 in that a hyperuricemia model was constructed with a different method.

1. Therapeutic effect of mUox@iLAND in model animals

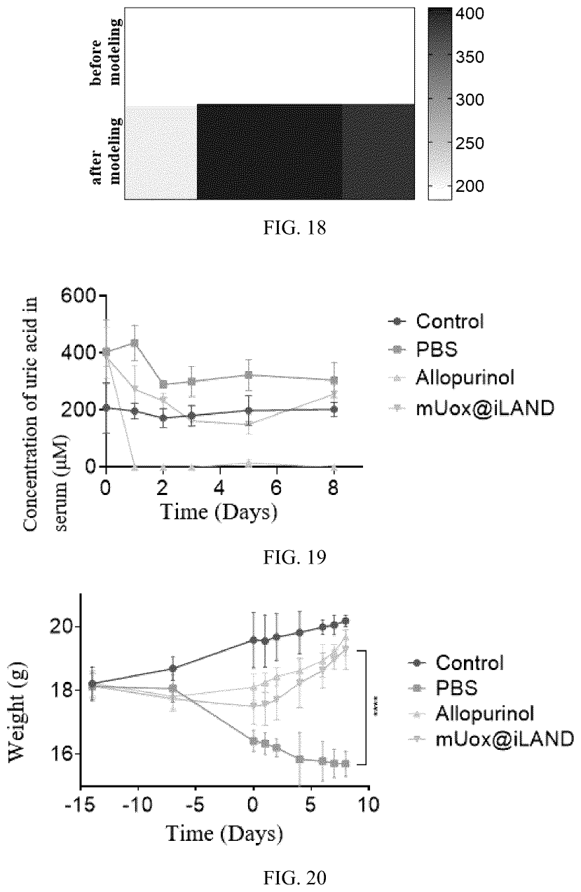

Female C57BL/6 mice aged 6 to 8 weeks were purchased from SPF (BEIJING) BIOTECHNOLOGY CO., LTD. The mice were randomly divided into 4 groups and subjected to different treatments, i.e., one group was fed normally, and the other three groups were constructed as models of hyperuricemia. For the model mice, the mice were fed with 0.15% high-purine feed starting from Day −14, that is, the hyperuricemia model was constructed by continuous high-purine diet. On Day 0, blood of all the mice was collected by orbital sampling, and serum was separated to detect uric acid levels. After confirming that the models of mice were successfully modeled, the mice were administrated with drug. The mice that were not subjected to the model construction were raised normally and administrated through tail vein injection with 200 μL of PBS (Control group, G1). The mice were subject to the model construction, and on 14 days after the model construction, the mice were administrated through tail vein injection with 200 μL of PBS (PBS group, G2). The mice were subject to the model construction, and on 14 days after the model construction, a certain amount of Allopurinol (which is a first-line clinical drug for anti-hyperuricemia, used as an experimental positive control group) was added to the drinking water, and the mice were allowed to drink the drinking water freely every day (Allopurinol group, G3). The mice were subject to the model construction, and on 14 days after the model construction, the mice were administrated through tail vein injection with 200 μL of mUox@iLAND prepared in Example 2 (mUox@iLAND group, G4), which was only administered once. The drugs were administered on the first day. Except for the Allopurinol group, which was administered daily, the other three groups were administered once during the whole course. At different time points after the drug administration, blood of the mice was collected by orbital sampling, and the serum was separated and analyzed for uric acid and other biochemical indices. The changes in weight of the mice were recorded throughout the model construction and drug administration. At the end of the experiment, the eyeballs were removed to collect blood and the mice were killed. The liver tissue was taken for metabolomics analysis, and the heart, liver, spleen, lung, kidney, and other tissues and organs were fixed and analyzed for pathological changes by H&E staining. The test results were shown in FIG. 18.

The results of model construction were shown in FIG. 18. Through the 14 days of model construction, the uric acid level in the model mice was significantly increased (from 100 μM to 400 μM), indicating that the hyperuricemia mouse model was successfully constructed.

The anti-hyperuricemia treatment results were shown in FIG. 19. Compared with the PBS group, the clinical first-line drug, Allopurinol, lowered the uric acid level below the normal level, even to around 0 μM, indicating that Allopurinol has certain risks in application and thus is not conducive to maintaining uric acid in human body at a normal level. For the mUox@iLAND group, the mice were only needed to be administered once to maintain uric acid in the mice at the normal level, indicating that lipid nanoparticles mUox@iLAND can achieve safe and efficient treatment of hyperuricemia, exhibiting more significant advantages.

The changes in weight of the mice in each group were shown in FIG. 20. The weight of the mice in the mUox@iLAND group was similar to that in the Control group, indicating that mUox@iLAND has good safety.

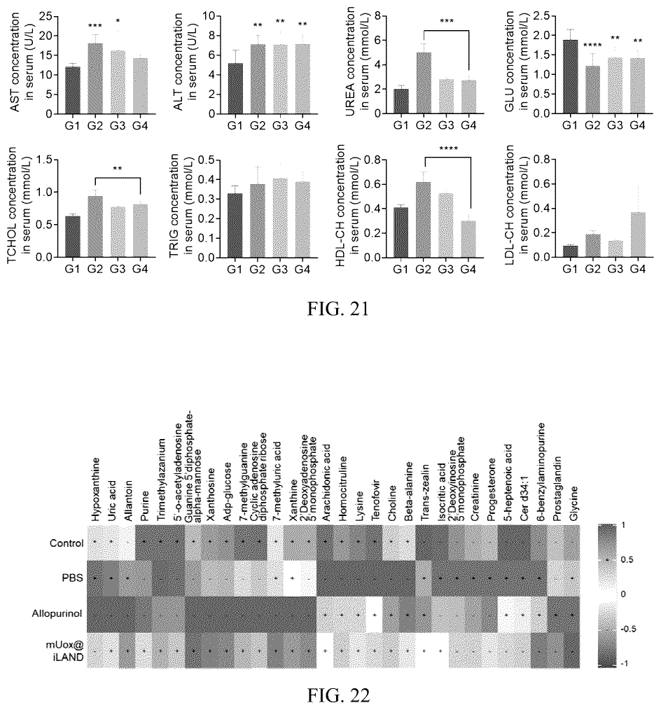

The serum biochemical results shown in FIG. 21, and mUox@iLAND showed no obvious toxic side effects.

The results of metabolomics analysis in FIG. 22. The metabolomics results indicate that mUox@iLAND mediated excellent anti-hyperuricemia effects, and the molecular metabolic levels in the treated mice were closest to those in the healthy mice Control group.

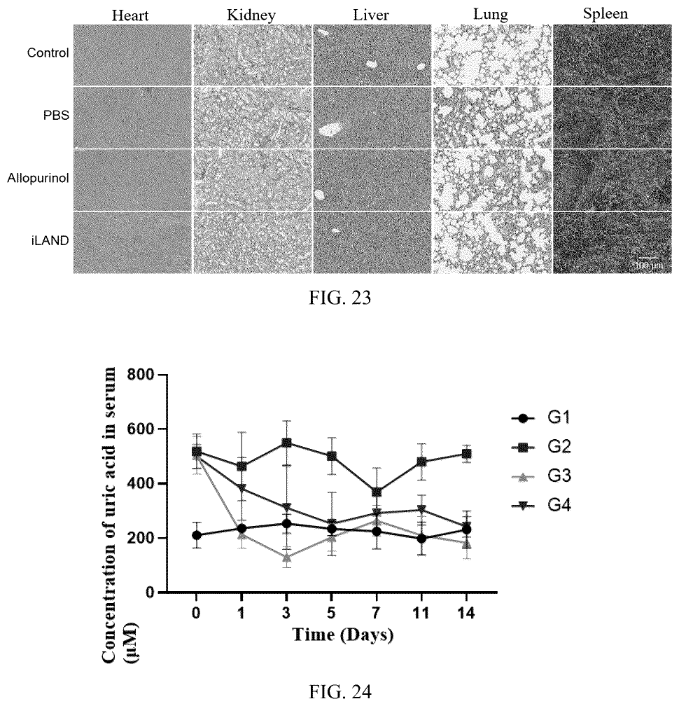

The pathological section results in FIG. 23 indicate that mUox@iLAND has good safety.

In summary, mUox@iLAND can stabilize the uric acid level for a long period of time by only once administration, and has superior safety and efficacy over the first-line clinical drug Allopurinol. Thus, mUox@iLAND provides a new approach for the research and development of the clinical drugs.

2. Therapeutic effect of Uox1@iLAND in model animals

The same method as step 1 of the present example was used to construct the hyperuricemia mouse model, and the therapeutic effect of the novel lipid nanoparticle Uox1@iLAND in the hyperuricemia mouse model was evaluated. The mice were randomly divided into 4 groups and subjected to different treatments. The mice in group G1 were not subjected to the model construction, raised normally, and administrated through tail vein injection with 200 μL of PBS (Control group). On 14 days after the model construction, the mice in group G2 were administered through tail vein injection with 200 μL of PBS (PBS group). On 14 days after the model construction, the mice in group G3 were administered through tail vein injection with 200 μL of PBS (PBS group). On 14 days after the model construction, a certain amount of Allopurinol (which is a first-line clinical drug for anti-hyperuricemia, used as an experimental positive control group) was added to the drinking water, and the mice in group G3 were allowed to drink the drinking water freely every day. On 14 days after the model construction, the mice in group G4 were administered through tail vein injection with 200 μL of Uox1@iLAND prepared in Example 2 (Uox1@iLAND group), which was administered only once. At different time points after the drug administration, blood of the mice was collected by orbital sampling, and the serum was separated and the uric acid level was measured.

The experimental results were shown in FIG. 24. Through the 14 days of model construction, the uric acid level in the model mice (G2 group) was significantly increased compared with the unmodeled animals (G1 group), indicating that the hyperuricemia mouse model was successfully constructed. The results of the positive control group (G3 group) indicate that the clinical first-line drug Allopurinol can lower the uric acid level to a level same as the normal level or a level lower than the normal level, while Uox1@iLAND in the G4 group can maintain the uric acid in the mice at the normal level by only once administration, indicating that the lipid nanoparticles Uox1@iLAND can achieve safe and efficient treatment of hyperuricemia.

In summary, Uox1@iLAND can stabilize the uric acid level for a long period of time by only once administration, indicating that the novel Uox protein and lipid nanoparticles designed in the present disclosure can be used to regulate the uric acid levels, providing a new approach for the research and development of the clinical drugs.

### Example 10

Female C57BL/6 mice aged 6 to 8 weeks were purchased from SPF (BEIJING) BIOTECHNOLOGY CO., LTD. The mice were randomly divided into 4 groups and subjected to different treatments, i.e., one group was fed normally, and the other three groups were constructed as models of hyperuricemia. For the model mice, the mice were fed with 0.15% high-purine feed starting from Day −14, that is, the hyperuricemia model was constructed by continuous high-purine diet. On Day 0, blood of all the mice was collected by orbital sampling, and serum was separated to detect uric acid levels. After confirming that the models of mice were successfully modeled, the mice were administrated with drug. The mice that were not subjected to the model construction were raised normally and administrated through tail vein injection with 200 μL of PBS (Control group, G1). The mice were subject to the model construction, and on 14 days after the model construction, the mice were administrated through tail vein injection with 200 μL of PBS (PBS group, G2). The mice were subject to the model construction, and on 14 days after the model construction, the mice were administrated through tail vein injection with 200 μL of mUox prepared in Example 1 (mUox, G3), which was only administered once. The mice were subject to the model construction, and on 14 days after the model construction, the mice were administrated through tail vein injection with 200 μL of mUox@iLAND prepared in Example 2 (mUox@iLAND group, G4), which was only administered once. The drugs were administered on the first day. The blood of the mice was collected by orbital sampling and the uric acid level in the serum of the mice was measured on Day 2 (24 hours after the administration), Day 3 (48 hours after the administration), and Day 7 (144 hours after the administration). The results of model construction and anti-hyperuricemia treatment were shown in Table 2. Through the 14 days of model construction, the uric acid level in the model mice was significantly increased (from 100 μM to 400 μM), indicating that the hyperuricemia mouse model was successfully constructed. The anti-hyperuricemia treatment results reveal that compared with the PBS group, the mUox@iLAND group can significantly reduce the uric acid level in the mice, while the uric acid level in the mice in the Free mUox group did not change significantly before and after the administration, indicating that mUoxmRNA alone without the novel lipid nanoparticles as a delivery carrier cannot achieve targeted delivery and protein expression in vivo.

TABLE 2

| | Concentration of uric acid (μM) | | | | |
|---|---|---|---|---|---|
| Group | −14 days | 0 days | 2 days | 3 days | 7 days |
| Control | 178 | 217 | 186 | 193 | 207 |
| PBS | 169 | 504 | 489 | 452 | 475 |
| mUox@iLAND | 174 | 500 | 285 | 236 | 199 |
| Free mUox | 186 | 502 | 497 | 464 | 487 |

### Example 11

1) A1-D1-5, cholesterol, DOPE, and DMG-PEG 2000 were taken and dissolved in anhydrous ethanol to form a 2 mg/mL organic phase solution.

2) An organic phase was prepared according to a molar ratio of A1-D1-5, DOPE, cholesterol, and DMG-PEG 2000 of 30:10:45:0.5, to obtain Organic Phase 1. An organic phase was prepared according to the molar ratio of A1-D1-5, DOPE, cholesterol, and DMG-PEG 2000 of 70:10:45:0.5 to obtain Organic Phase 2. An organic phase was prepared according to the molar ratio of A1-D1-5, DOPE, cholesterol, and DMG-PEG 2000 of 30:60:45:0.5 to obtain Organic Phase 3. An organic phase was prepared according to the molar ratio of A1-D1-5, DOPE, cholesterol, and DMG-PEG 2000 of 30:10:80:0.5 to obtain Organic Phase 4. An organic phase was prepared according to the molar ratio of A1-D1-5, DOPE, cholesterol, and DMG-PEG 2000 of 30:10:45:5 to obtain Organic Phase 5.

3) The solutions of the Organic Phase 1 to Organic Phase 5 were injected into 3-fold volume of 50 mM sodium citrate buffer solution (pH=4.0) at a high speed, while stirred at a high speed to form iLAND1, iLAND2, iLAND3, iLAND4, and iLAND5.

4) The mRNAs (mUox) obtained in Example 1 were mixed with equal volumes of five iLANDs (the mass ratio of iLAND to mRNA was 15:1) and incubated at 5020 C. for 10 minutes.

5) The incubated solution was transferred to a 100 kD dialysis bag and dialyzed in 1×PBS for 2 hours to obtain mUox@iLAND1, mUox@iLAND2, mUox@iLAND3, mUox@iLAND4, and mUox@iLAND5.

6) The five types of mUox@iLAND were transfected into Hepa1-6 cells. Total protein of the cells was extracted after 24 hours of the transfection, and the expression level of mUox was detected by Western blotting.

The experimental results in Table 3 reveal that mUox@iLAND1 can achieve efficient expression of the protein, and its expression level was calculated as 100%. The other four liposomes are unable to achieve efficient expression of the protein, indicating that the specific composition ratio of the lipids in the present disclosure is the key to achieving efficient expression of mRNA.

TABLE 3

| | Molar ratio | | | | |
|---|---|---|---|---|---|
| | A1- | | | DMG- | Expression |
| | D1- | | | PEG- | level of |
| Group | 5 | DOPE | Cholesterol | 2000 | protein |
| Mock | — | — | — | — | 0% |
| mUox@iLAND1 | 30 | 10 | 45 | 0.5 | 100% |
| mUox@iLAND2 | 70 | 10 | 45 | 0.5 | 0.1% |
| mUox@iLAND3 | 30 | 60 | 45 | 0.5 | 61.9% |
| mUox@iLAND4 | 30 | 10 | 80 | 0.5 | 0.1% |
| mUox@iLAND5 | 30 | 10 | 45 | 5 | 0% |

Although embodiments of the present disclosure are illustrated and described above, it can be understood that the above embodiments are illustrative and should not be construed as limitations of the present disclosure. Those skilled in the art can make changes, modifications, substitutions, and variations to the above embodiments within the scope of the present disclosure.

What is claimed is:

1. An mRNA-liposome complex, comprising:
a liposome; and
a nucleic acid, wherein:
the nucleic acid comprises at least one mRNA encoding urate oxidase or recombinant urate oxidase;
the liposome comprises at least two lipids, the at least two lipids comprising a core lipid, and a molar percentage of the core lipid in a total molar amount of the liposome being not less than 15%;
the liposome has a pKa ranging from 6.0 to 6.3;
the mRNA comprises a nucleotide sequence as set forth in any one of SEQ ID No: 7 to SEQ ID No: 12, or a nucleotide sequence having at least 90% sequence similarity to the nucleotide sequence as set forth in any one of SEQ ID No: 7 to SEQ ID No: 12; and
a weight ratio of the liposome to the mRNA is (1 to 30):1.

2. The mRNA-liposome complex according to claim 1, wherein:
the liposome comprises the core lipid, an auxiliary lipid, a steroid, and a PEG lipid; and
a molar ratio of the core lipid, the auxiliary lipid, the steroid, and the PEG lipid is (20 to 60):(10 to 50):(30 to 50):(0.5 to 2.5).

3. The mRNA-liposome complex according to claim 2, wherein the molar ratio of the core lipid, the auxiliary lipid, the steroid, and the PEG lipid is (30 to 39):(9 to 15):(40 to 50):(0.5 to 1).

4. The mRNA-liposome complex according to claim 2, wherein the auxiliary lipid is selected from at least one of 1,2-distearoyl-sn-glycero-3-phosphocholine (DSPC), 1,2-dipalmitoyl-sn-glycero-3-phosphocholine (DPPC), 1,2-dipalmitoyl-sn-glycero-3-phosphoethanolamine (DPPE), 1,2-dimyristoyl-sn-glycero-3-phosphocholine (DMPC), 1,2-dimyristoyl-sn-glycero-3-phosphoethanolamine (DMPE), 1-palmitoyl-2-oleoyl-sn-glycero-3-phosphocholine (POPC), 1,2-dioleoyl-sn-glycero-3-phosphocholine (DOPC), 2-dioleoyl-sn-glycerol-3-phospho-(1′-rac-glycerol) (DOPG), 1,2-dioleoyl-sn-glycero-3-phosphoethanolamine (DOPE), sphingomyelin (SM), and diethyl pyrocarbonate (DEPC).

5. The mRNA-liposome complex according to claim 2, wherein the steroid is selected from at least one of cholesterol, coprosterol, sitosterol, ergosterol, campesterol, stigmasterol, and brassicasterol, and preferably, cholesterol.

6. The mRNA-liposome complex according to claim 2, wherein the PEG lipid is selected from at least one of 2-[(polyethylene glycol)-2000]-N,N-tetracosyl acetamide (ALC-0159), 1,2-dimyristoyl-sn-glycero-methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmitoyl, PEG-dioleyl, PEG-distearyl, PEG-diacylglycerolamide (PEG-DAG), PEG-dipalmitoyl phosphatidylethanolamine (PEG-DPPE), PEG-phosphatidylethanolamine (PEG-PE), PEG-succinic diacylglycerol (PEG-S-DAG), PEG-ceramide (PEG-cer), PEG-dialkoxypropyl carbamate, and PEG-1,2-dimyristoyloxypropyl-3-amine (PEG-c-DMA).

7. The mRNA-liposome complex according to claim 2, wherein the PEG lipid is selected from at least one of DMG-PEG 2000, DSPE-PEG 2000, DPPE-PEG 2000, and DMA-PEG 2000.

8. The mRNA-liposome complex according to claim 1, wherein the core lipid is selected from a compound having a structure represented by Formula (I) or a stereoisomer, tautomer, solvate, and pharmaceutically acceptable salt thereof,

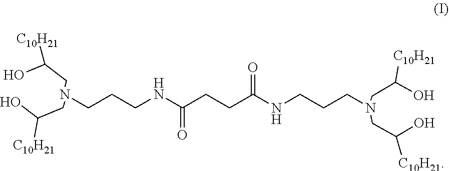

9. The mRNA-liposome complex according to claim 1, wherein the urate oxidase or recombinant urate oxidase satisfies at least one of the following conditions:
the urate oxidase or recombinant urate oxidase is mainly expressed in the liver;
the urate oxidase or recombinant urate oxidase has activity in reducing uric acid level; and
the recombinant urate oxidase or urate oxidase comprises an amino acid sequence as set forth in SEQ ID No: 1 or SEQ ID NO: 4, or an amino acid sequence having at least 90% sequence homology to the amino acid sequence as set forth in SEQ ID No: 1 or SEQ ID NO: 4.

10. The mRNA-liposome complex according to claim 1, wherein:
5′ end of the mRNA is linked to 5′UTR; and/or
3′ end of the mRNA is linked to 3′UTR.

11. The mRNA-liposome complex according to claim 1, wherein the weight ratio of the liposome to the mRNA is (10 to 20):1.

12. A method for preparing the mRNA-liposome complex according to claim 1, the method comprising:
mixing the liposome with the nucleic acid, to obtain the mRNA-liposome complex.

13. The method according to claim 12, wherein:
the liposome is obtained by: dissolving each of a core lipid, an auxiliary lipid, a steroid, and a PEG lipid with an organic solvent, and obtaining a core lipid solution, an auxiliary lipid solution, a steroid solution, and a PEG lipid solution; and mixing the core lipid solution, the auxiliary lipid solution, the steroid solution, and the PEG lipid solution in a first buffer, to obtain the liposome; and
optionally, the organic solvent is an alcohol solvent;
optionally, the alcohol solvent is selected from C₁ to C₄ alcohol solvent;
optionally, the alcohol solvent is selected from at least one of methanol, ethanol, propanol, and butanol, and preferably, the alcohol solvent is selected from methanol and ethanol, and more preferably, the alcohol solvent is ethanol; and
optionally, the first buffer is selected from at least one of a sodium citrate buffer, an acetate buffer, and a sodium bicarbonate buffer.

14. The method according to claim 12, further comprising, prior to said mixing the liposome with the nucleic acid:
dissolving the mRNA with a second buffer, wherein:
optionally, the second buffer is selected from at least one of a sodium citrate buffer, an acetate buffer, and a sodium bicarbonate buffer; and
optionally, the second buffer further comprises an ethanol aqueous solution with a volume percentage concentration ranging from 20% to 30%.

15. A pharmaceutical composition, comprising:
the mRNA-liposome complex according to claim 1, and
a pharmaceutically acceptable excipient or carrier.

16. A method for treating and/or preventing hyperuricemia-related diseases, comprising:
administering a pharmaceutically acceptable amount of the mRNA-liposome complex according to claim 1 to a subject; wherein:
optionally, the hyperuricemia-related diseases are selected from at least one of hyperuricemia, gout, gouty nephropathy, gouty vasculopathy, and gouty cardiomyopathy; and
optionally, an administration route of the mRNA-liposome complex is subcutaneous injection or intravenous injection.


SEQUENCE LISTING (14 sequences)

SEQ ID NO: 1 - protein, 303 aa, Mus musculus
MAHYHDNYGKNDEVEFVRTGYGKDMVKVLHIQRDGKYHSIKEVATSVQLTLRSKKDYLHGDNSDIIPTDTIKNTVHVLAKLRGIRNIETFAMNICEHFLSSFNHVTRAHVYVEEVPWKRFEKNGIKHVHAFIHTPTGTHFCEVEQMRNGPPVIHSGIKDLKVLKTTQSGFEGFLKDQFTTLPEVKDRCFATQVYCKWRYQRRDVDFEAIWGAVRDIVLQKFAGPYDKGEYSPSVQKTLYDIQVLSLSQLPEIEDMEISLPNIHYFNIDMSKMGLINKEEVLLPLDNPYGKITGTVKRKLPSRL

SEQ ID NO: 2 - protein, 304 aa, Macaca mulatta
MADYHNNYKKNDELEFVRTGYGKDMVKVLHIQRDGKYHSIKEVATSVQLTLSSKKDYVHGDNSDIIPTDTIKNTVHVLAKLKGIKSIEAFGVNICEYFLSSFNHVIRAQVYVEEIPWKRLEKNGVKHVHAFIHTPTGTHFCEVEQLRSGPPVIHSGIKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 3 - protein, 302 aa, synthetic construct
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 4 - protein, 304 aa, synthetic construct
MAHYRNTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 5 - protein, 305 aa, synthetic construct
MAHYHNNYKKNDEVEFVRTGYGKEMVKVLHIQRDGKYHSIKEVATSVQLTLSSKKDYLHGDNSDIIPTDTIKNTVHVLAKFKEIKSIEAFGVNICEHFLSSFNHVIRAQVYMEEIPWKHLGKNGVKHVHAFIHTPTGTHFCEVEQLRSGPQVIHSGIKDLKVLKTTQSGFEGFIKDQFTTLPEVKDRCFATQVYCKWRYHQCRDVDFKATWDTIRDLVMEKSAGPYDKGEYLTSVQKTLCDIQVLSLSRVPAIEDMEISLPNIHYFNIDMSKMGLINKEEVRGLAAIRQSIWKNYWYSQEEVVFK

SEQ ID NO: 6 - protein, 304 aa, synthetic construct
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVIHSGIKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 7 - DNA, 912 bp, Mus musculus
  source 1..912: mol_type = other DNA
atggcccattaccatgacaactacggaaagaatgatgaagtggaatttgtccgaactggctatgggaaagacatggtcaaagttctccatattcagagagatggaaaataccacagcatcaaagaggtggcaacttcggtccagttgactctgaggtccaagaaggattacctacacggtgataattccgacatcatccctacagacaccatcaagaacactgtgcacgtcctggcgaagctcagagggatcagaaacatcgagacctttgcaatgaacatctgtgagcacttcctctcttcttttaaccatgtcactcgagcccacgtctacgtggaagaggtcccctggaaacgatttgaaaagaatgggatcaaacacgtccatgcattcatccacaccccgacgggaacgcacttctgtgaggtggagcagatgagaaacggacctcccgtcattcactctggaatcaaagacctcaaggtcttgaaaacaacccagtctgggtttgaagggttcctcaaggaccagttcactaccctccctgaggtgaaggaccgatgctttgccactcaagtgtactgcaagtggcgctaccagagacgggacgtggacttcgaggctatctggggcgctgtccgggacattgtcctgcagaaatttgctgggccctatgacaaaggtgaatactcaccttccgtgcagaagaccctctatgatatacaagtgctgtccctgagccagcttcctgagatagaagacatggaaatcagccttccgaacattcactactttaacatcgacatgtccaaaatgggactgatcaacaaggaagaggttttgctgcctctcgacaatccctacggcaaaataacagggacagtcaagaggaagctgccttccaggctgtga

SEQ ID NO: 8 - DNA, 915 bp, Macaca mulatta
  source 1..915: mol_type = other DNA
atggccgactaccataacaactataaaaagaatgatgagttggagtttgtccgaactggctatgggaaggatatggtaaaagttctccatattcagcgagatggaaaatatcacagcattaaagaggtggcaacttcagtgcaacttactctgagttccaaaaaagattacgtgcatggagataattcagatatcatccctacagacaccatcaagaacacagttcatgtcttggcaaagttgaagggaatcaaaagcatagaagcctttggtgtgaatatttgtgagtattttctttcttcttttaaccatgtaatccgagctcaagtctacgtggaagaaatcccttggaagcgtcttgaaaagaatggagttaagcatgtccatgcatttattcacactcccactggaacacacttctgtgaagttgaacaactgagaagtggaccccccgtcattcattctggaatcaaagacctcaaggtcttgaaaacaacacagtctggatttgaaggtttcatcaaggaccagttcaccaccctccctgaggtgaaggaccgatgctttgccacccaagtgtactgcaagtggcgctaccaccagtgcagggatgtggacttcgaggctacctggggcaccattcgggaccttgtcctggagaaatttgctgggccctatgacaaaggcgagtactcgccctctgtgcagaagaccctctatgatatacaggtgctctccctgagccgagttcctgagatagaagatatggaaatcagcctgccaaacattcactacttcaatatagacatgtccaaaatgggtctgatcaacaaggaagaggtcttgctgccattagacaatccatatggaaaaattactggtacagtcaagaggaagttgtcttcaagactgtga

SEQ ID NO: 9 - DNA, 909 bp, synthetic construct
  source 1..909: mol_type = other DNA
atgagcgccgtgaaagcggcccgttatggcaaagataatgtccgcgtgtacaaagttcataaagacgaaaaaaccggcgttcagacggtctatgaaatgaccgtttgcgtcctgctggaaggtgaaattgaaacctcatacacgaaagcggataactcggtgattgttgccaccgacagcatcaaaaacaccatttatatcacggcgaaacagaatccggtgaccccgccggaactgtttggcagtattctgggtacgcacttcatcgaaaaatacaaccatattcacgcagctcatgtgaatatcgtttgtcaccgttggacccgcatggatattgacggcaaaccgcatccgcacagttttatccgtgattccgaagaaaaacgcaacgtccaggtggatgtggttgaaggcaaaggtattgacatcaaaagctctctgtctggcctgaccgtgctgaaaagcaccaacagccaattttggggtttcctgcgtgatgaatataccacgctgaaagaaacctgggaccgcattctgagcaccgatgttgacgcgacgtggcagtggaaaaatttttctggtctgcaagaagtccgtagtcatgtgccgaaattcgatgcaacctgggcgacggcccgcgaagttaccctgaaaacgttcgctgaagacaactcagcatcggtccaggctaccatgtacaaaatggcagaacaaatcctggctcgtcagcaactgattgaaacggtggaatattccctgccgaacaaacattacttcgaaattgatctgtcatggcacaaaggcctgcagaacaccggtaaaaatgcggaagtcttcgccccgcaaagcgatccgaacggcctgatcaaatgcaccgttggtcgcagttccctgaaaagcaaactgtga

SEQ ID NO: 10 - DNA, 915 bp, synthetic construct
  source 1..915: mol_type = other DNA
atggctcattaccgtaatacgtacaaaaagaatgatgaggtagagtttgtccgaactggctatgggaaggatatgataaaagttctccatattcagcgagatggaaaatatcacagcattaaagaggtggcaactacagtgcaactgactttgagctccaaaaaagattacctgcatggagacaattcagatgtcatccctacagacaccatcaagaacacagttaatgtcctggcgaagttcaaaggcatcaaaagcatagaaacttttgctgtgactatctgtgagcatttcctttcttccttcaagcatgtcatcagagctcaagtctatgtggaagaagttccttggaagcgttttgaaaagaatggagttaagcatgtccatgcatttatttatactcctactggaacgcacttctgtgaggttgaacagataaggaatggacctccagtcattcattctggaatcaaagacctaaaagtcttgaaaacaacccagtctggctttgaaggattcatcaaggaccagttcaccaccctccctgaggtgaaggaccggtgctttgccacccaagtgtactgcaaatggcgctaccaccagggcagagatgtggactttgaggccacctgggacactgttaggagcattgtcctgcagaaatttgctgggccctatgacaaaggcgagtactcgccctctgtccagaagacactctatgacatccaggtgctcaccctgggccaggttcctgagatagaagatatggaaatcagcctgccaaatattcactacttaaacatagacatgtccaaaatgggactgatcaacaaggaagaggtcttgctacctttagacaatccatatggcaagattactggtacagtcaagaggaagctgtcttcaaggctgtga

SEQ ID NO: 11 - DNA, 918 bp, synthetic construct
  source 1..918: mol_type = other DNA
atggcccactaccataacaactataaaaagaatgatgaggtggagtttgtccgaactggctatgggaaggaaatggtaaaagttctccatattcagcgagatggaaaatatcacagcattaaagaggtggcaacttcagtgcaacttactctaagttccaaaaaagattacctgcatggagataattcagacatcatccctacagacaccatcaagaacacagttcatgtcttggcaaagtttaaagaaatcaaaagcatagaagcctttggtgtgaatatttgtgagcattttctttcttcttttaaccatgtaatccgagctcaagtctacatggaagaaatcccttggaagcatcttggaaagaatggagttaagcatgtccatgcatttattcacactcccactggaacacacttctgtgaagttgaacagctgagaagtggaccccaagtcattcattctggaatcaaagacctcaaggtcttgaaaacaacacagtctggatttgaaggtttcatcaaggaccagttcactaccctccctgaggtgaaggaccgatgctttgccacccaagtgtactgcaagtggcgctaccaccagtgcagggatgtggacttcaaggctacctgggacaccattcgggaccttgtcatggagaaatctgctgggccctatgacaaaggtgaatacttgacctctgtgcagaagaccctctgtgatatccaggtgctctccctgagccgagttcctgcgatagaagatatggaaatcagcctgccaaacattcactacttcaacatagacatgtccaaaatgggtctgatcaacaaggaagaggtaagaggtcttgctgccattagacaatccatatggaaaaattactggtacagtcaagaggaagttgtcttcaagtga

SEQ ID NO: 12 - DNA, 915 bp, synthetic construct
  source 1..915: mol_type = other DNA
atggccgactaccataacaactataaaaagaatgatgagttggagtttgtccgaactggctatgggaaggatatggtaaaagttctccatattcagcgagatggaaaatatcacagcattaaagaggtggcaacttcagtgcaacttactctgagttccaaaaaagattacctgcatggagataattcagatatcatccctacagacaccatcaagaacacagttcatgtcttggcaaagtttaagggaatcaaaagcatagaagcctttggtgtgaatatttgtgagtattttctttcttcttttaaccatgtaatccgagctcaagtctacgtggaagaaatcccttggaagcgtcttgaaaagaatggagttaagcatgtccatgcatttattcacactcccactggaacacacttctgtgaagttgaacaactgagaagtggaccccccgtcattcattctggaatcaaagacctcaaggtcttgaaaacaacacagtctggatttgaaggtttcatcaaggaccagttcaccaccctccctgaggtgaaggaccgatgctttgccacccaagtgtactgcaagtggcgctaccaccagtgcagggatgtggacttcgaggctacctggggcaccattcgggaccttgtcctggagaaatttgctgggccctatgacaaaggcgagtactcgccctctgtgcagaagaccctctatgatatacaggtgctctccctgagccgagttcctgagatagaagatatggaaatcagcctgccaaacattcactacttcaatatagacatgtccaaaatgggtctgatcaacaaggaagaggtcttgctgccattagacaatccatatggaaaaattactggtacagtcaagaggaagttgtcttcaagactgtga

SEQ ID NO: 13 - DNA, 155 bp, synthetic construct
  source 1..155: mol_type = other DNA
tcaagcttttggaccctcgtacagaagctaatacgactcactatagggaaataagagagaaaagaagagtaagaagaaatataagagccaccatggagacagacacactcctgctatgggtactgctgctctgggttccaggttccactggtgac

SEQ ID NO: 14 - DNA, 119 bp, synthetic construct
  source 1..119: mol_type = other DNA
tgataataggctggagcctcggtggccatgcttcttgccccttgggcctccccccagcccctcctccccttcctgcacccgtacccccgtggtctttgaataaagtctgagtgggcggc